(19) United States
(12) Patent Application Publication
(10) Pub. No.: US 2023/0330267 A1
(43) Pub. Date: Oct. 19, 2023

(54) NOVEL ENGINEERED CAPSID SEROTYPE OF RECOMBINANT ADENO-ASSOCIATED VIRAL VECTOR WITH ENHANCED TRANSDUCTION EFFICIENCY AND WIDESPREAD DISTRIBUTION IN THE BRAIN

(71) Applicant: Ohio State Innovation Foundation, Columbus, OH (US)

(72) Inventors: Lei Cao, Columbus, OH (US); Wei Huang, Columbus, OH (US); Krzysztof Bankiewicz, Columbus, OH (US); Piotr Hadaczek, Columbus, OH (US); Lluis Samaranch, Columbus, OH (US)

(21) Appl. No.: 18/024,806

(22) PCT Filed: Sep. 3, 2021
(86) PCT No.: PCT/US2021/049081

§ 371 Date: Mar. 6, 2023

Related U.S. Application Data
(60) Provisional application No. 63/074,548, filed on Sep. 4, 2020.

(51) Int. Cl. A61K 48/00; A61K 38/18; C12N 15/86; A61K 38/20; A61K 38/30

(52) U.S. Cl. CPC A61K 48/0058; A61K 38/1808; A61K 38/1825; A61K 38/1841; A61K 38/185; A61K 38/1858; A61K 38/1866; A61K 38/1875; A61K 38/2066; A61K 38/30; C12N 15/86; C12N 2750/14122; C12N 2750/14143; C12N 2750/14145; C12N 2750/14152; C12N 2750/14171

(57) ABSTRACT

Disclosed are engineered brain tropic adeno-associated viral vectors and methods of their use.

### I. CROSS REFERENCE TO RELATED APPLICATIONS

1. This application claims the benefit of U.S. Provisional Pat. Application Serial No. 63/074,548 filed Sep. 4, 2020, the disclosure of which is expressly incorporated herein by reference in its entirety.

### II. BACKGROUND

2. Adeno-associated viral (AAV) vectors are often used in gene therapy for neurological disorders because of its safety profile and promising results in clinical trials. One challenge to AAV-based gene therapy is effective transduction of large numbers of the appropriate cell type. The brain and neural tissue have been a particularly challenging site for effective transduction. What are needed are new vectors that can effectively and efficiently transduce neural tissue and specifically the brain.

### III. SUMMARY

3. Disclosed are engineered adeno-associated viral vectors and uses thereof.

4. In one aspect, disclosed herein are engineered adeno-associated virus (AAV) vectors comprising a recombinant 2 (Rec2) capsid with one or more substitutions, insertions, and/or deletions in the heparin binding loci (for example a substitution, deletion, or and/or insertion at a residue corresponding to a residue between residues 561 and 591 of SEQ ID NO: 1) wherein the substitution confers neuronal tropism to the vector. In one aspect, the one or more substitutions occurs at a residue corresponding to residues 585, 587, 588, 589, and/or 594 of SEQ ID NO: 1 (such as, for example, a substitution such as Q588P, Q589L, Q589I, Q589V, Q589G, Q594L, Q5941, and/or Q594V).

5. Also, disclosed herein are engineered AAV vectors of any preceding aspect, further comprising a first expression cassette comprising a regulatory element (such as, for example, a woodchuck posttranscriptional regulatory element (WPRE) sequence) and a transgene (such as, for example, β-Galactosidase 1 (GLB1), Niemann-Pick C1 (NPC1), Apolipoprotein E (APOE), GD3 synthase, huntingtin (Htt), interleukin (IL)-10 (IL-10), Myelin Oligodendrocyte Glycoprotein (MOG), mitogen-activated protein kinase 8 interacting protein 3 (MAPKA8IP3), survival motor neuron (SMN) 1 (SMN1), SMN2, Cas9, β-Glucocerebrosidase (GBA), Sphingomyelin phosphodiesterase 1 (SMPD1), beta-hexosaminidase A (HEXA), nerve growth factor (NGF), brain-derived neurotrophic factor (BDNF), neurotrophin (NT) 3 (NT-3), NT-⅘, NT-6, Glial Cell Derived Neurotrophic Factor (GDNF), Ciliary Neurotrophic Factor (CNTF), Leukemia inhibitory factor (LIF), Insulin-like growth factor (IGF) 1 (IGF-1), β-fibroblast growth factor (FGF), neurturin, persephin, artemin, transforming growth factor (TGF) alpha (TGFα), TGFβ, IGF-2, platelet derived growth factor (PDGF), epidermal growth factor (EGF), cardiotropin, vascular endothelial growth factor (VEGF), Sonic hedgehog (SHH), bone morphogenic proteins (BMP), FGF20, Vasoactive Intestinal Peptide (VIP), pleiotrophin (PTN), Aromatic L-amino Acid Decarboxylase (AADC), TH, 5-hydroxytryptamine (5HT), hepatocyte growth factor (HGF), miRNA-222, miRNA-7, and/or miRNA-132) operatively linked to a promoter and a second expression cassette comprising a tissue specific promoter operatively linked to a RNA silencing element that targets the regulatory element in the first expression cassette.

6. In one aspect, disclosed herein are methods of delivering a gene (such as, for example, β-Galactosidase 1 (GLB1), Niemann-Pick C1 (NPC1), Apolipoprotein E (APOE), GD3 synthase, huntingtin (Htt), interleukin (IL)-10 (IL-10), Myelin Oligodendrocyte Glycoprotein (MOG), mitogen-activated protein kinase 8 interacting protein 3 (MAPKA8IP3), survival motor neuron (SMN) 1 (SMN1), SMN2, Cas9, β-Glucocerebrosidase (GBA), Sphingomyelin phosphodiesterase 1 (SMPD1), beta-hexosaminidase A (HEXA), nerve growth factor (NGF), brain-derived neurotrophic factor (BDNF), neurotrophin (NT) 3 (NT-3), NT-⅘, NT-6, Glial Cell Derived Neurotrophic Factor (GDNF), Ciliary Neurotrophic Factor (CNTF), Leukemia inhibitory factor (LIF), Insulin-like growth factor (IGF) 1 (IGF-1), β-fibroblast growth factor (FGF), neurturin, persephin, artemin, transforming growth factor (TGF) alpha (TGFα), TGFβ, IGF-2, platelet derived growth factor (PDGF), epidermal growth factor (EGF), cardiotropin, vascular endothelial growth factor (VEGF), Sonic hedgehog (SHH), bone morphogenic proteins (BMP), FGF20, Vasoactive Intestinal Peptide (VIP), pleiotrophin (PTN), Aromatic L-amino Acid Decarboxylase (AADC), TH, 5-hydroxytryptamine (5HT), hepatocyte growth factor (HGF), miRNA-222, miRNA-7, and/or miRNA-132) to neural tissue in the brain (for example the frontal and/or prefrontal cortex) of a subject comprising administering to the subject the engineered AAV vector of any preceding aspect. In some aspects, the AAV vector has at least a 50, 51, 52, 53, 54, 55, 56, 57, 58, 59, 60, 61, 62, 63, 64, 65, 66,67, 68, 69, 70, 71, 72, 73, 74, 75% efficiency of transduction. Also disclosed are methods of delivering a gene to neural tissue wherein the AAV vector is administered systemically (such as, for example, intravenously, including but not limited to, i.v. injection or i.v. drip; and/or retro-orbitally); or via cerebrospinal fluid injection.

7. Also disclosed herein are methods of treating, inhibiting, decreasing, reducing, ameliorating, and/or preventing a neurological disease (such as, for example, Alzheimer’s disease, Parkinson’s disease, Multiple Systems Atrophy (MSA), Lysosomal Storage Disease (LSD), and/or muscular dystrophy) in a subject comprising administering to the subject the engineered AAV vector of any preceding aspect encoding a therapeutic agent (such as, for example a transgene including, but not limited to β-Galactosidase 1 (GLB1), Niemann-Pick C1 (NPC1), Apolipoprotein E (APOE), GD3 synthase, huntingtin (Htt), interleukin (IL)-10 (IL-10), Myelin Oligodendrocyte Glycoprotein (MOG), mitogen-activated protein kinase 8 interacting protein 3 (MAPKA8IP3), survival motor neuron (SMN) 1 (SMN1), SMN2, Cas9, β-Glucocerebrosidase (GBA), Sphingomyelin phosphodiesterase 1 (SMPD1), beta-hexosaminidase A (HEXA), nerve growth factor (NGF), brain-derived neurotrophic factor (BDNF), neurotrophin (NT) 3 (NT-3), NT-⅘, NT-6, Glial Cell Derived Neurotrophic Factor (GDNF), Ciliary Neurotrophic Factor (CNTF), Leukemia inhibitory factor (LIF), Insulin-like growth factor (IGF) 1 (IGF-1), β-fibroblast growth factor (FGF), neurturin, persephin, artemin, transforming growth factor (TGF) alpha (TGFa), TGFβ, IGF-2, platelet derived growth factor (PDGF), epidermal growth factor (EGF), cardiotropin, vascular endothelial growth factor (VEGF), Sonic hedgehog (SHH), bone morphogenic proteins (BMP), FGF20, Vasoactive Intestinal Peptide (VIP), pleiotrophin (PTN), Aromatic L-amino Acid Decarboxylase (AADC), TH, 5-hydroxytryptamine (5HT), hepatocyte growth factor (HGF), miRNA-222, miRNA-7, and/or miRNA-132). Also disclosed are methods of treating, inhibiting, decreasing, reducing, ameliorating, and/or preventing a neurological disease of any preceding aspect, wherein the AAV vector is administered systemically (such as, for example, intravenously, including but not limited to, i.v. injection or i.v. drip; retro-orbitally; or via cerebrospinal fluid injection).

### IV. BRIEF DESCRIPTION OF THE DRAWINGS

8. The accompanying drawings, which are incorporated in and constitute a part of this specification, illustrate several embodiments and together with the description illustrate the disclosed compositions and methods.

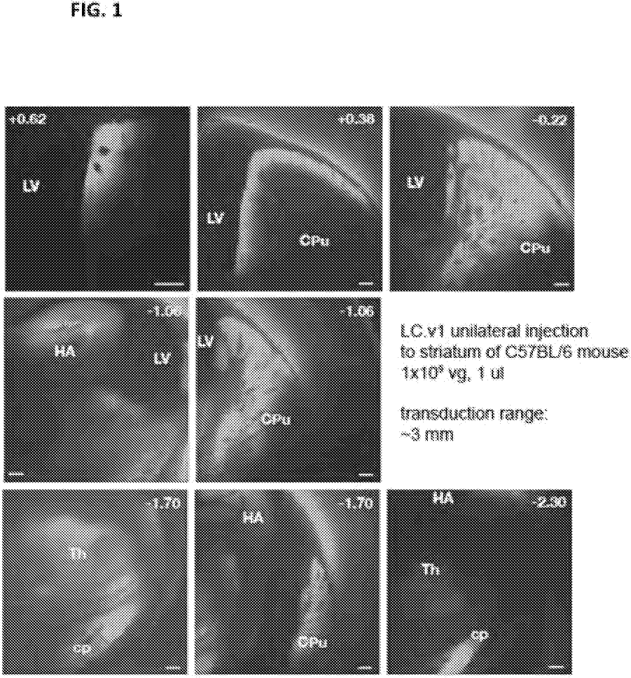

9. FIG. 1 shows a representative GFP fluorescence 3 weeks post unilateral injection of LC. V1 vector to the striatum of mouse.

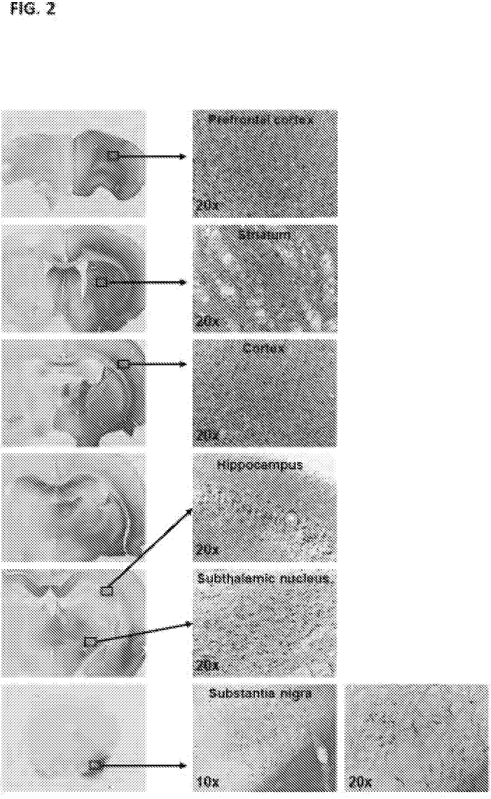

10. FIG. 2 shows that rat 1 transduced with AAV:LC.V1 (lot# LC-195); Titer: 1.2×10¹³ vg/ml. Delivery was CED into the right striatum (15 ul; 1 ul/min). Euthanasia was performed 3 weeks after the transduction. Staining was IHC against GFP (transgene).

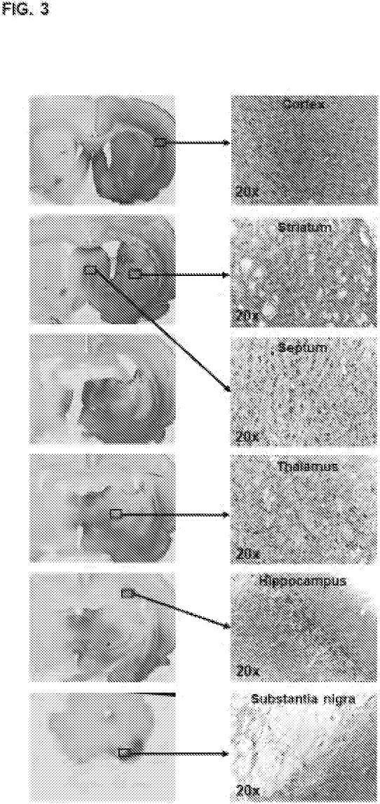

11. FIG. 3 shows rat 2 transduced with AAV: LC.V1 (lot# LC-195); Titer: 1.2×10¹³ vg/ml. Delivery was CED into the right striatum (15 ul; 1 ul/min). Euthanasia was performed 3 weeks after the transduction. Staining was IHC against GFP (transgene).

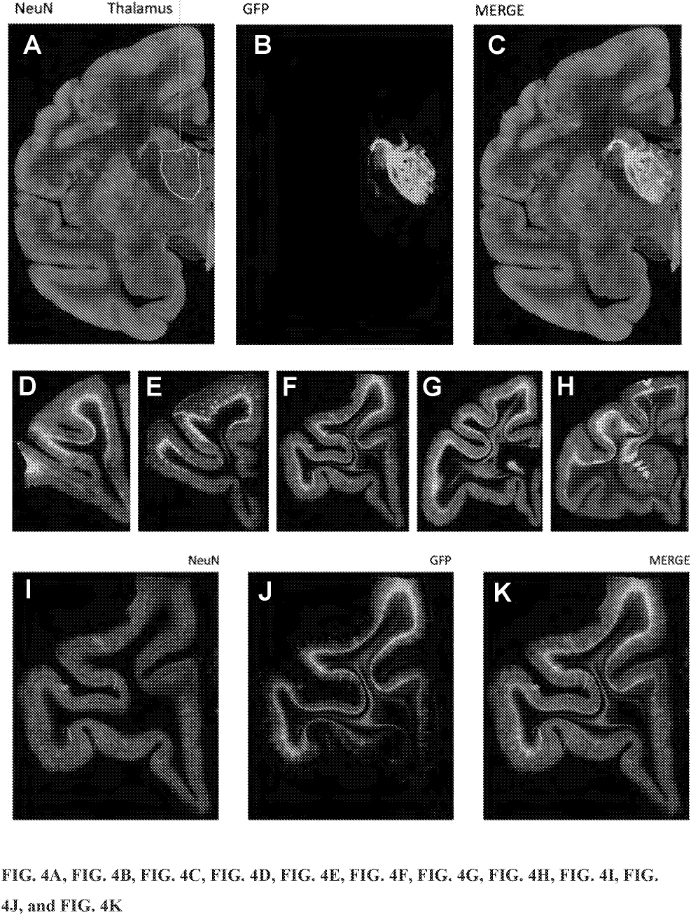

12. FIGS. 4A-4C show intracerebral delivery of LC.V1 into the thalamus of a non-human primate (NHP). 4A - NHP brain section stained (red fluorescence) with neuronal marker, NeuN. 4B - NHP brain section stained (green fluorescence) against transgene/reporter gene, GFP. 4C - merged staining of NeuN + GFP showing near complete coverage of the monkey thalamus.

13. FIGS. 4D-4K show fluorescence staining of NHP brain sections showing transduction of pyramidal neurons of layer V within the prefrontal and frontal cortex. This transduction was the result of retrograde transport of LC.V1 from the site of injection (thalamus). Red fluorescence - staining against a neuronal marker, NeuN; green fluorescence -reporter gene, GFP.

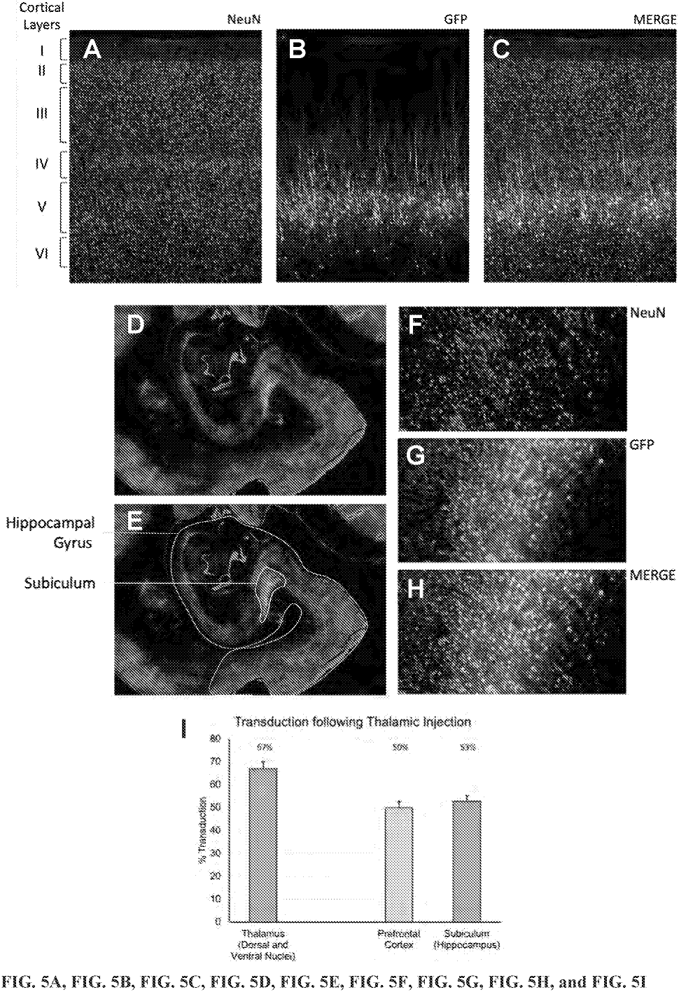

14. FIGS. 5A-5C show a higher magnification of a representative NHP stained (double fluorescence) brain section from the prefrontal cortex showing transduction of neurons within layer V. 5A - brain section was stained against a neuronal marker, NeuN (red fluorescence). 5B – rain section was stained against a reporter gene, GFP (green fluorescence). 5Cmerged staining of NeuN + GFP.

15. FIGS. 5D-5H show neuronal transduction of hippocampus/subiculum by LC.V1 vector injected into the thalamus (primary site of injection) - the result of retrograde transport from thalamus. The NHP sections were stained against a neuronal marker, NeuN (red fluorescence) and a reporter gene, GFP (green fluorescence). A higher magnification of the brain section from that area is shown in panels 5F-5H.

16. FIG. 5I shows the efficiency of neuronal transduction with LC.V1 vector injected into the NHP thalamus. The values were calculated from brain sections stained by double fluorescence against NeuN and GFP.

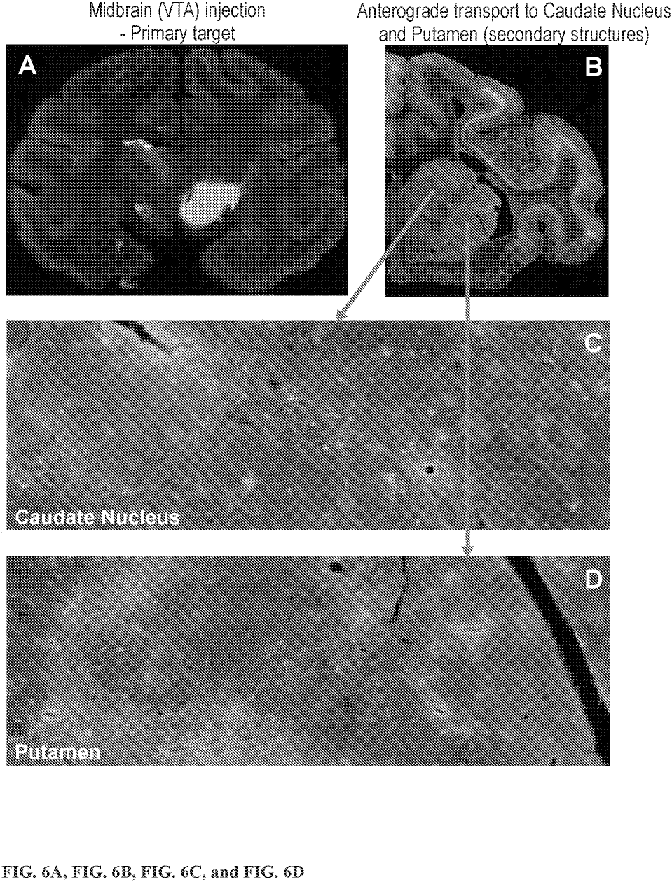

17. FIGS. 6A-6D show neuronal transduction of the primary target - midbrain (VTA and substantia nigra) with LC.V1 vector (6A) and its anterograde transport to distant brain structures, caudate nucleus (6B and 6C) and putamen (6B and 6D). The NHP sections were stained against a neuronal marker, NeuN (red fluorescence) and a reporter gene, GFP (green fluorescence).

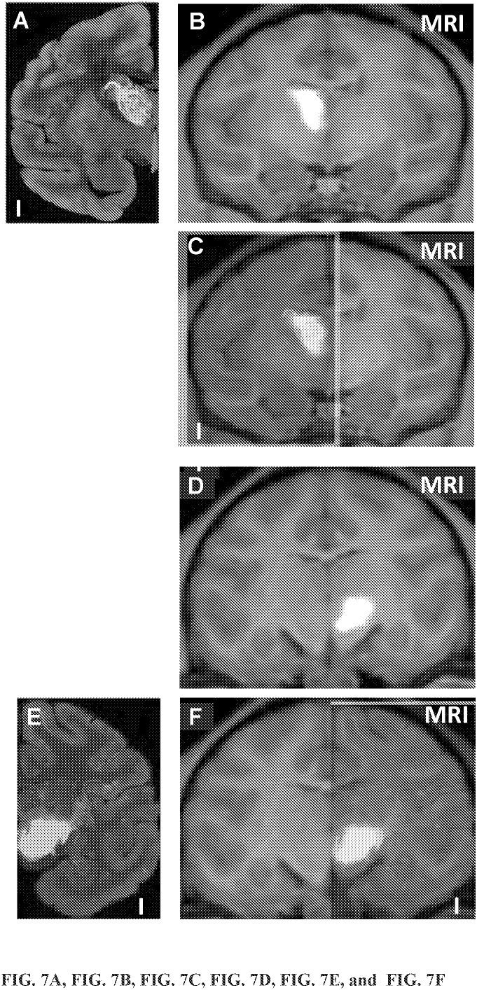

18. FIGS. 7A-7F show that distribution of LC.V1 vector within the brain parenchyma can be monitored by real-time MRI. 7A - an NHP thalamic brain section stained with double fluorescence against a neuronal marker, NeuN (red fluorescence) and a reporter gene, GFP (green fluorescence). 7B - an MRI scan from a real-time MRI during the injection of LC.V1 mixed with the MRI contrast agent, ProHance. 7C - superimposed FIGS. 7A and 7B showing a near-perfect correlation of GFP expression from LC.V1 vector and area of ProHance signal. 7D an MRI scan from a real-time MRI during the injection of LC.V1 mixed with the MRI contrast agent, ProHance into the right midbrain (VTA and substantia nigra). 7E - an NHP brain section stained with double fluorescence against a neuronal marker, NeuN (red fluorescence) and a reporter gene, GFP (green fluorescence). 7F - superimposed FIGS. 7D and 7E showing a near-perfect correlation of GFP expression from LC.V1 vector and area of ProHance signal.

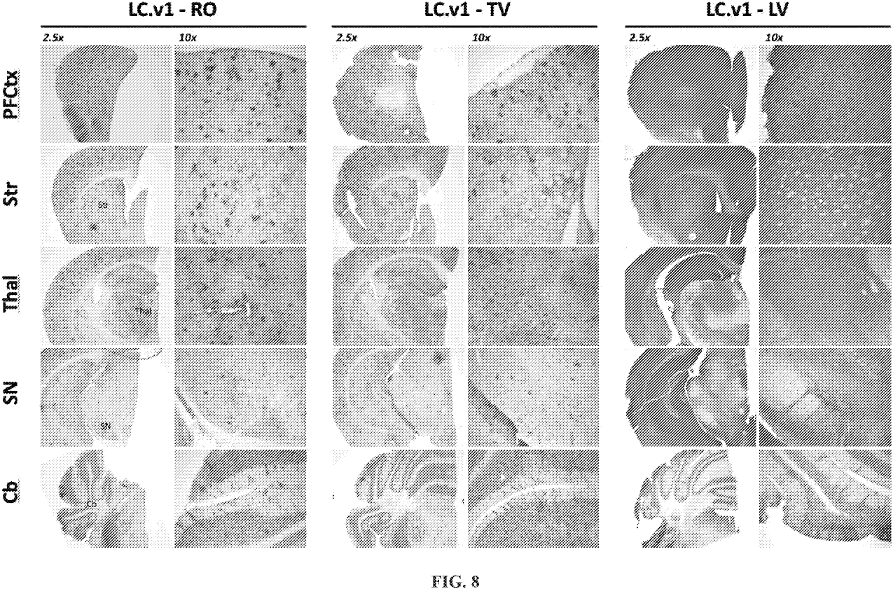

19. FIG. 8 shows representative images of transgene transduction (GFP) from representative animals at different brain levels from anterior-posterior axis. Animals received either systemic injection throughout right retro-orbital sinus (RO) or Tail vein (TV), or received a CSF injection through the left lateral ventricle delivery (LV). Each animals received 50 µL systemically or 25 µL into the CSF of AAV:LC.V1 at a titer of 8.36E+13 vg/mL (lot# CS1851) Immunohistochemistry against GFP (transgene) 1:1000 with a counter staining of Cresyl Violet. Abbreviations: Pre-frontal cortex (PFCtx), Striatum (Str), Thalamus (Thal), Substantia Nigra (SN), Cerebellum (Cb), Green fluorescent protein (GFP).

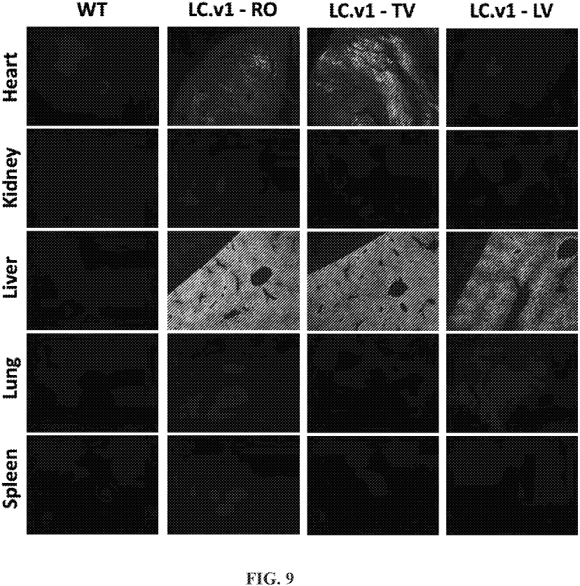

20. FIG. 9 shows representative images of transgene transduction (GFP) in different peripheral organs from representative animals. Animals received either systemic injection throughout right retro-orbital sinus (RO) or Tail vein (TV), or received a CSF injection through the left lateral ventricle delivery (LV). Each animals received 50 µL systemically or 25 µL into the CSF of AAV:LC.V1 at a titer of 8.36E+13 vg/mL (lot# CS1851). Immunohistochemistry against GFP (transgene) 1:1000 with a counter staining of Cresyl Violet. A naive mouse (uninjected) was included as negative control to show absence of GFP signal. Green signal shows native GFP signal from vector transduction. Abbreviations: Wild type (WT), Green fluorescent protein (GFP).

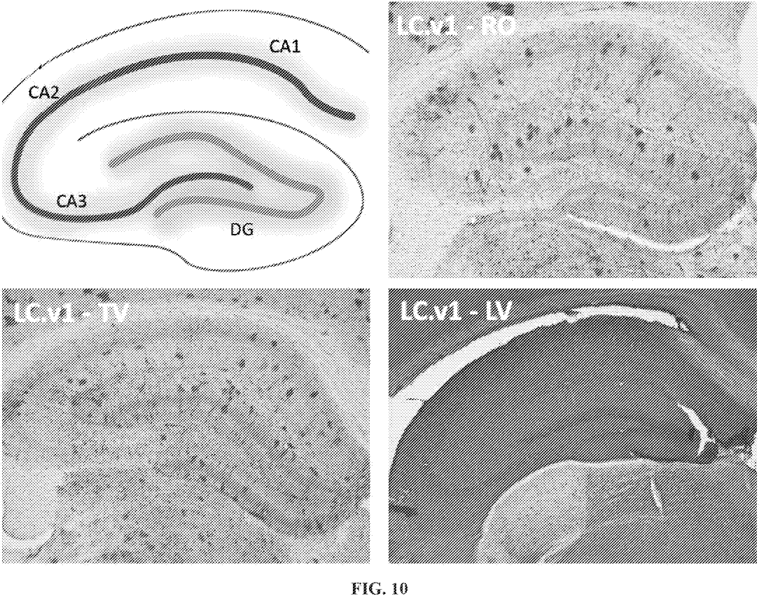

21. FIG. 10 shows representative images of transgene transduction (GFP) in hippocampus and dentate gyrus from representative animals. Animals received either systemic injection throughout right retro-orbital sinus (RO) or Tail vein (TV), or received a CSF injection through the left lateral ventricle delivery (LV). Each animals received 50 µL systemically or 25 µL into the CSF of AAV:LC.V1 at a titer of 8.36E+13 vg/mL (lot# CS1851). Immunohistochemistry against GFP (transgene) 1:1000 with a counter staining of Cresyl Violet Abbreviations: Hippocampal cornu ammonis (CA), Dentate gyrus (DG), Green fluorescent protein (GFP).

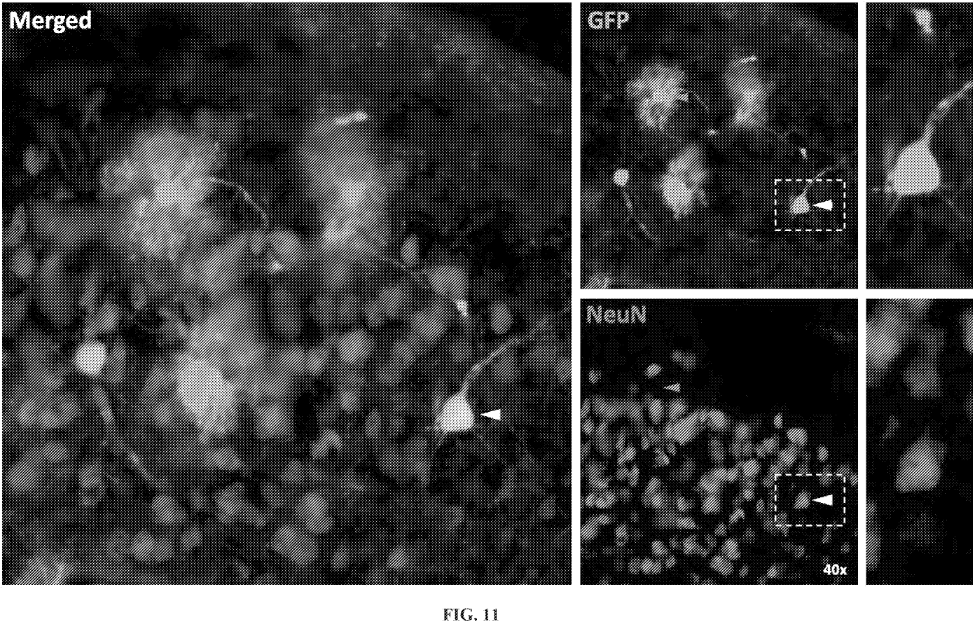

22. FIG. 11 shows representative image for neuronal tropism assessment (NeuN). Double immunofluorescence staining against GFP (transgene, green) and the specific neuronal marker NeuN (red). White arrow heads indicate NeuN/GFP colocalization confirming the neuronal tropism of LC.V1 vector. Blue arrow heads show non-NeuN cells transduced. Abbreviations: Green fluorescent protein (GFP), Neuronal Nuclei protein (NeuN)

### V. DETAILED DESCRIPTION

23. Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### A. Definitions

24. As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

25. Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10”as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

26. In this specification and in the claims which follow, reference will be made to a number of terms which shall be defined to have the following meanings:

27. “Optional” or “optionally” means that the subsequently described event or circumstance may or may not occur, and that the description includes instances where said event or circumstance occurs and instances where it does not.

28. An “increase” can refer to any change that results in a greater amount of a symptom, disease, composition, condition or activity. An increase can be any individual, median, or average increase in a condition, symptom, activity, composition in a statistically significant amount. Thus, the increase can be a 1, 2, 3, 4, 5, 6, 7, 8, 9, 10, 15, 20, 25, 30, 35, 40, 45, 50, 55, 60, 65, 70, 75, 80, 85, 90, 95, or 100% increase so long as the increase is statistically significant.

29. A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also, for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

30. “Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

31. By “reduce” or other forms of the word, such as “reducing” or “reduction,” is meant lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control.

32. By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

33. The term “subject” refers to any individual who is the target of administration or treatment. The subject can be a vertebrate, for example, a mammal. In one aspect, the subject can be human, non-human primate, bovine, equine, porcine, canine, or feline. The subject can also be a guinea pig, rat, hamster, rabbit, mouse, or mole. Thus, the subject can be a human or veterinary patient. The term “patient” refers to a subject under the treatment of a clinician, e.g., physician.

34. The term “therapeutically effective” refers to the amount of the composition used is of sufficient quantity to ameliorate one or more causes or symptoms of a disease or disorder. Such amelioration only requires a reduction or alteration, not necessarily elimination.

35. The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder, and supportive treatment, that is, treatment employed to supplement another specific therapy directed toward the improvement of the associated disease, pathological condition, or disorder.

36. “Biocompatible” generally refers to a material and any metabolites or degradation products thereof that are generally non-toxic to the recipient and do not cause significant adverse effects to the subject.

37. “Comprising” is intended to mean that the compositions, methods, etc. include the recited elements, but do not exclude others. “Consisting essentially of” when used to define compositions and methods, shall mean including the recited elements, but excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions provided and/or claimed in this disclosure. Embodiments defined by each of these transition terms are within the scope of this disclosure.

38. A “control” is an alternative subject or sample used in an experiment for comparison purposes. A control can be “positive” or “negative.”

39. “Effective amount” of an agent refers to a sufficient amount of an agent to provide a desired effect. The amount of agent that is “effective” will vary from subject to subject, depending on many factors such as the age and general condition of the subject, the particular agent or agents, and the like. Thus, it is not always possible to specify a quantified “effective amount.” However, an appropriate “effective amount” in any subject case may be determined by one of ordinary skill in the art using routine experimentation. Also, as used herein, and unless specifically stated otherwise, an “effective amount” of an agent can also refer to an amount covering both therapeutically effective amounts and prophylactically effective amounts. An “effective amount” of an agent necessary to achieve a therapeutic effect may vary according to factors such as the age, sex, and weight of the subject. Dosage regimens can be adjusted to provide the optimum therapeutic response. For example, several divided doses may be administered daily or the dose may be proportionally reduced as indicated by the exigencies of the therapeutic situation.

40. A “pharmaceutically acceptable” component can refer to a component that is not biologically or otherwise undesirable, i.e., the component may be incorporated into a pharmaceutical formulation provided by the disclosure and administered to a subject as described herein without causing significant undesirable biological effects or interacting in a deleterious manner with any of the other components of the formulation in which it is contained. When used in reference to administration to a human, the term generally implies the component has met the required standards of toxicological and manufacturing testing or that it is included on the Inactive Ingredient Guide prepared by the U.S. Food and Drug Administration.

41. “Pharmaceutically acceptable carrier” (sometimes referred to as a “carrier”) means a carrier or excipient that is useful in preparing a pharmaceutical or therapeutic composition that is generally safe and non-toxic and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

42. “Pharmacologically active” (or simply “active”), as in a “pharmacologically active” derivative or analog, can refer to a derivative or analog (e.g., a salt, ester, amide, conjugate, metabolite, isomer, fragment, etc.) having the same type of pharmacological activity as the parent compound and approximately equivalent in degree.

43. “Therapeutic agent” refers to any composition that has a beneficial biological effect. Beneficial biological effects include both therapeutic effects, e.g., treatment of a disorder or other undesirable physiological condition, and prophylactic effects, e.g., prevention of a disorder or other undesirable physiological condition (e.g., a non-immunogenic cancer). The terms also encompass pharmaceutically acceptable, pharmacologically active derivatives of beneficial agents specifically mentioned herein, including, but not limited to, salts, esters, amides, proagents, active metabolites, isomers, fragments, analogs, and the like. When the terms “therapeutic agent” is used, then, or when a particular agent is specifically identified, it is to be understood that the term includes the agent per se as well as pharmaceutically acceptable, pharmacologically active salts, esters, amides, proagents, conjugates, active metabolites, isomers, fragments, analogs, etc.

44. “Therapeutically effective amount” or “therapeutically effective dose” of a composition (e.g. a composition comprising an agent) refers to an amount that is effective to achieve a desired therapeutic result. In some embodiments, a desired therapeutic result is the control of a neurological disease or disorder. Therapeutically effective amounts of a given therapeutic agent will typically vary with respect to factors such as the type and severity of the disorder or disease being treated and the age, gender, and weight of the subject. The term can also refer to an amount of a therapeutic agent, or a rate of delivery of a therapeutic agent (e.g., amount over time), effective to facilitate a desired therapeutic effect, such as pain relief. The precise desired therapeutic effect will vary according to the condition to be treated, the tolerance of the subject, the agent and/or agent formulation to be administered (e.g., the potency of the therapeutic agent, the concentration of agent in the formulation, and the like), and a variety of other factors that are appreciated by those of ordinary skill in the art. In some instances, a desired biological or medical response is achieved following administration of multiple dosages of the composition to the subject over a period of days, weeks, or years.

45. Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### B. Compositions

46. Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular engineered adeno-associated viral vector is disclosed and discussed and a number of modifications that can be made to a number of molecules including the engineered adeno-associated viral vector are discussed, specifically contemplated is each and every combination and permutation of engineered adeno-associated viral vector and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

47. Improving the efficiency of viral gene delivery has become increasingly important to increase the success of various clinical trials. Structural domains of different rAAV serotypes isolated from primate brain were combined to create novel hybrid recombinant AAV serotype, rAAV2/rec2. However, Rec2 serotype was found no better than the naturally occurring AAV2 capsid regarding retinal transduction. Rec2 capsid serotype transduces adipose tissue with far superior efficiency than naturally occurring AAV serotypes. One concern of the Rec2 capsid is its high efficiency for liver transduction that hampers needs of selective gene transfer of the adipose tissue. In order to generate new serotypes with improved adipose-tropism and eliminating liver transduction, several point mutations were made in the capsid of Rec2 by analyzing amino acid sequence of capsid among AAV8, Rec2, Rec3, and AAV2.

48. In one aspect, disclosed herein are engineered adeno-associated virus (AAV) vectors comprising recombinant 2 (Rec2) capsid comprising one or more substitutions, insertions, and/or deletions in the heparin binding loci (for example a substitution, deletion, or and/or insertion at residues corresponding to a residue between residues 561 and 591 of the Rec2 capsid protein as set forth in SEQ ID NO: 1) wherein the substitution confers tropism for neural tissue to the vector. In one aspect, the one or more substitutions occurs at a residue corresponding to residues 585, 587, 588, 589, and/or 594 of SEQ ID NO: 1 (such as, for example, a substitution such as Q588P, Q589L, Q589I, Q589V, Q589G, Q594L, Q594I, and/or Q594V). In one aspect, the disclosed herein are engineered adeno-associated virus (AAV) vectors comprising recombinant 2 (Rec2) capsid comprising Q588P, Q589L, and Q594L substitution relative to SEQ ID NO: 1 and as shown in SEQ ID NO: 6.

49. Also, disclosed herein are engineered AAV vectors, further comprising a first expression cassette comprising a regulatory element (such as, for example, a woodchuck posttranscriptional regulatory element (WPRE) sequence) and a transgene operatively linked to a promoter. In one aspect, the AAV vector can also comprise a second cassette, wherein the second cassette comprises a tissue specific promoter operatively linked to a RNA silencing element that targets the regulatory element in the first expression cassette.

### 1. Homology/Identity

50. It is understood that one way to define any known variants and derivatives or those that might arise, of the disclosed genes and proteins herein is through defining the variants and derivatives in terms of homology to specific known sequences. Specifically disclosed are variants of these and other genes and proteins herein disclosed which have at least, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 percent homology to the stated sequence. Those of skill in the art readily understand how to determine the homology of two proteins or nucleic acids, such as genes. For example, the homology can be calculated after aligning the two sequences so that the homology is at its highest level.

51. Another way of calculating homology can be performed by published algorithms. Optimal alignment of sequences for comparison may be conducted by the local homology algorithm of Smith and Waterman Adv. Appl. Math. 2: 482 (1981), by the homology alignment algorithm of Needleman and Wunsch, J. Mol Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, WI), or by inspection.

52. The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989 which are herein incorporated by reference for at least material related to nucleic acid alignment.

### 2. Delivery of the Compositions to Cells

53. There are a number of compositions and methods which can be used to deliver nucleic acids to cells, either in vitro or in vivo. These methods and compositions can largely be broken down into two classes: viral based delivery systems and non-viral based delivery systems. For example, the nucleic acids can be delivered through a number of direct delivery systems such as, electroporation, lipofection, calcium phosphate precipitation, plasmids, viral vectors, viral nucleic acids, phage nucleic acids, phages, cosmids, or via transfer of genetic material in cells or carriers such as cationic liposomes. Appropriate means for transfection, including viral vectors, chemical transfectants, or physico-mechanical methods such as electroporation and direct diffusion of DNA, are described by, for example, Wolff, J. A., et al., Science, 247, 1465-1468, (1990); and Wolff, J. A. Nature, 352, 815-818, (1991). Such methods are well known in the art and readily adaptable for use with the compositions and methods described herein. In certain cases, the methods will be modified to specifically function with large DNA molecules. Further, these methods can be used to target certain diseases and cell populations by using the targeting characteristics of the carrier.

### A) Nucleic Acid Based Delivery Systems

54. Transfer vectors can be any nucleotide construction used to deliver genes into cells (e.g., a plasmid), or as part of a general strategy to deliver genes, e.g., as part of recombinant retrovirus or adenovirus (Ram et al. Cancer Res. 53:83-88, (1993)).

55. As used herein, viral vectors are agents that transport a transgene into a cell (such as, for example a brain cell) without degradation and include a promoter yielding expression of the gene in the cells into which it is delivered. In some embodiments the engineered viral vectors disclosed herein are derived from adeno-associated vectors (AAV).

### Adeno-Associated Viral Vectors

56. Another type of viral vector is based on an adeno-associated virus (AAV). This defective parvovirus is a preferred vector because it can infect many cell types and is nonpathogenic to humans. AAV type vectors can transport about 4 to 5 kb of DNA and wild type AAV is known to stably insert into chromosome 19 (such as, for example at AAV integration site 1 (AAVS1)). In one aspect, the disclosed AAV vector can comprise a selectable marker such as, for example, the gene encoding the green fluorescent protein, GFP.

57. In one aspect, the AAV contains a pair of inverted terminal repeats (ITRs) which flank at least one cassette containing a promoter which directs cell-specific expression operably linked to a heterologous gene. Heterologous in this context refers to any nucleotide sequence or gene which is not native to the AAV or B19 parvovirus.

58. Typically, the AAV and B19 coding regions have been deleted, resulting in a safe, noncytotoxic vector. The AAV ITRs (typically from AAV2), or modifications thereof, confer infectivity and site-specific integration, but not cytotoxicity, and the promoter directs cell-specific expression. U.S Pat. No. 6,261,834 is herein incorporated by reference for material related to the AAV vector.

59. The disclosed vectors thus are designed to persist in the nucleus as extrachromosomal and not integrate into the human genome. This ability to persist without integration adds to the safety of these vectors.

60. The inserted genes in viral and retroviral usually contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and may contain upstream elements and response elements.

### B) In Vivo/Ex Vivo

61. As described above, the compositions can be administered in a pharmaceutically acceptable carrier and can be delivered to the subject’s cells in vivo and/or ex vivo by a variety of mechanisms well known in the art (e.g., uptake of naked DNA, liposome fusion, intramuscular injection of DNA via a gene gun, endocytosis and the like).

62. If ex vivo methods are employed, cells or tissues can be removed and maintained outside the body according to standard protocols well known in the art. The compositions can be introduced into the cells via any gene transfer mechanism, such as, for example, calcium phosphate mediated gene delivery, electroporation, microinjection or proteoliposomes. The transduced cells can then be infused (e.g., in a pharmaceutically acceptable carrier) or homotopically transplanted back into the subject per standard methods for the cell or tissue type. Standard methods are known for transplantation or infusion of various cells into a subject.

### 3. Expression Systems

63. The nucleic acids that are delivered to cells typically contain expression controlling systems. For example, the inserted genes in viral and retroviral systems usually contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and may contain upstream elements and response elements.

### A) Viral Promoters and Enhancers

64. Preferred promoters controlling transcription from vectors in mammalian host cells may be obtained from various sources, for example, the genomes of viruses such as: polyoma, Simian Virus 40 (SV40), adenovirus, retroviruses, hepatitis-B virus and most preferably cytomegalovirus, or from heterologous mammalian promoters, e.g. beta actin promoter. The early and late promoters of the SV40 virus are conveniently obtained as an SV40 restriction fragment which also contains the SV40 viral origin of replication (Fiers et al., Nature, 273: 113 (1978)). The immediate early promoter of the human cytomegalovirus is conveniently obtained as a HindIII E restriction fragment (Greenway, P.J. et al., Gene 18: 355-360 (1982)). Of course, promoters from the host cell or related species also are useful herein.

65. Enhancer generally refers to a sequence of DNA that functions at no fixed distance from the transcription start site and can be either 5′ (Laimins, L. et al., Proc. Natl. Acad. Sci. 78: 993 (1981)) or 3′ (Lusky, M.L., et al., Mol. Cell Bio. 3: 1108 (1983)) to the transcription unit. Furthermore, enhancers can be within an intron (Banerji, J.L. et al., Cell 33: 729 (1983)) as well as within the coding sequence itself (Osborne, T.F., et al., Mol. Cell Bio. 4: 1293 (1984)). They are usually between 10 and 300 bp in length, and they function in cis. Enhancers function to increase transcription from nearby promoters. Enhancers also often contain response elements that mediate the regulation of transcription. Promoters can also contain response elements that mediate the regulation of transcription. Enhancers often determine the regulation of expression of a gene. While many enhancer sequences are now known from mammalian genes (globin, elastase, albumin, -fetoprotein and insulin), typically one will use an enhancer from a eukaryotic cell virus for general expression. Preferred examples are the SV40 enhancer on the late side of the replication origin (bp 100-270), the cytomegalovirus early promoter enhancer, the polyoma enhancer on the late side of the replication origin, and adenovirus enhancers.

66. The promotor and/or enhancer may be specifically activated either by light or specific chemical events which trigger their function. Systems can be regulated by reagents such as tetracycline and dexamethasone. There are also ways to enhance viral vector gene expression by exposure to irradiation, such as gamma irradiation, or alkylating chemotherapy drugs.

67. In certain embodiments the promoter and/or enhancer region can act as a constitutive promoter and/or enhancer to maximize expression of the region of the transcription unit to be transcribed. In certain constructs the promoter and/or enhancer region be active in all eukaryotic cell types, even if it is only expressed in a particular type of cell at a particular time. A preferred promoter of this type is the CMV promoter (650 bases). Other preferred promoters are SV40 promoters, cytomegalovirus (full length promoter), and retroviral vector LTR.

68. It has been shown that all specific regulatory elements can be cloned and used to construct expression vectors that are selectively expressed in specific cell types such as melanoma cells. The glial fibrillary acetic protein (GFAP) promoter has been used to selectively express genes in cells of glial origin.

69. Expression vectors used in eukaryotic host cells (yeast, fungi, insect, plant, animal, human or nucleated cells) may also contain sequences necessary for the termination of transcription which may affect mRNA expression. These regions are transcribed as polyadenylated segments in the untranslated portion of the mRNA encoding tissue factor protein. The 3′ untranslated regions also include transcription termination sites. It is preferred that the transcription unit also contains a polyadenylation region. One benefit of this region is that it increases the likelihood that the transcribed unit will be processed and transported like mRNA. The identification and use of polyadenylation signals in expression constructs is well established. It is preferred that homologous polyadenylation signals be used in the transgene constructs. In certain transcription units, the polyadenylation region is derived from the SV40 early polyadenylation signal and consists of about 400 bases. It is also preferred that the transcribed units contain other standard sequences alone or in combination with the above sequences improve expression from, or stability of, the construct.

### B) Markers

70. The viral vectors can include nucleic acid sequence encoding a marker product. This marker product is used to determine if the gene has been delivered to the cell and once delivered is being expressed. Preferred marker genes are the E. Coli lacZ gene, which encodes ß-galactosidase, and green fluorescent protein.

71. In some embodiments the marker may be a selectable marker. Examples of suitable selectable markers for mammalian cells are dihydrofolate reductase (DHFR), thymidine kinase, neomycin, neomycin analog G418, hydromycin, and puromycin. When such selectable markers are successfully transferred into a mammalian host cell, the transformed mammalian host cell can survive if placed under selective pressure. There are two widely used distinct categories of selective regimes. The first category is based on a cell’s metabolism and the use of a mutant cell line which lacks the ability to grow independent of a supplemented media. Two examples are: CHO DHFR- cells and mouse LTK- cells. These cells lack the ability to grow without the addition of such nutrients as thymidine or hypoxanthine. Because these cells lack certain genes necessary for a complete nucleotide synthesis pathway, they cannot survive unless the missing nucleotides are provided in a supplemented media. An alternative to supplementing the media is to introduce an intact DHFR or TK gene into cells lacking the respective genes, thus altering their growth requirements. Individual cells which were not transformed with the DHFR or TK gene will not be capable of survival in non-supplemented media.

72. The second category is dominant selection which refers to a selection scheme used in any cell type and does not require the use of a mutant cell line. These schemes typically use a drug to arrest growth of a host cell. Those cells which have a novel gene would express a protein conveying drug resistance and would survive the selection. Examples of such dominant selection use the drugs neomycin, (Southern P. and Berg, P., J. Molec. Appl. Genet. 1: 327 (1982)), mycophenolic acid, (Mulligan, R.C. and Berg, P. Science 209: 1422 (1980)) or hygromycin, (Sugden, B. et al., Mol. Cell. Biol. 5: 410-413 (1985)). The three examples employ bacterial genes under eukaryotic control to convey resistance to the appropriate drug G418 or neomycin (geneticin), xgpt (mycophenolic acid) or hygromycin, respectively. Others include the neomycin analog G418 and puramycin.

### 4. Peptides

### A) Protein Variants

73. As discussed herein there are numerous variants of the AAV capsid proteins (VP1, VP2, and VP3) that are known and herein contemplated. In addition, to the known functional strain variants there are derivatives of the AAV capsid proteins which also function in the disclosed methods and compositions. Protein variants and derivatives are well understood to those of skill in the art and, in some instances, can involve amino acid sequence modifications. For example, amino acid sequence modifications typically fall into one or more of three classes: substitutional, insertional or deletional variants. Insertions include amino and/or carboxyl terminal fusions as well as intrasequence insertions of single or multiple amino acid residues. Insertions ordinarily will be smaller insertions than those of amino or carboxyl terminal fusions, for example, on the order of one to four residues. Immunogenic fusion protein derivatives, such as those described in the examples, are made by fusing a polypeptide sufficiently large to confer immunogenicity to the target sequence by cross-linking in vitro or by recombinant cell culture transformed with DNA encoding the fusion. Deletions are characterized by the removal of one or more amino acid residues from the protein sequence. Typically, no more than about from 2 to 6 residues are deleted at any one site within the protein molecule. These variants ordinarily are prepared by site specific mutagenesis of nucleotides in the DNA encoding the protein, thereby producing DNA encoding the variant, and thereafter expressing the DNA in recombinant cell culture. Techniques for making substitution mutations at predetermined sites in DNA having a known sequence are well known, for example M13 primer mutagenesis and PCR mutagenesis. Amino acid substitutions are typically of single residues, but can occur at a number of different locations at once; insertions usually will be on the order of about from 1 to 10 amino acid residues; and deletions will range about from 1 to 30 residues. Deletions or insertions preferably are made in adjacent pairs, i.e. a deletion of 2 residues or insertion of 2 residues. Substitutions, deletions, insertions or any combination thereof may be combined to arrive at a final construct. The mutations must not place the sequence out of reading frame and preferably will not create complementary regions that could produce secondary mRNA structure. Substitutional variants are those in which at least one residue has been removed and a different residue inserted in its place. Such substitutions generally are made in accordance with the following Tables 1 and 2 and are referred to as conservative substitutions.

TABLE 1

Amino Acid Abbreviations

| | | |
|---|---|---|
| Amino Acid | Abbreviations | |
| Alanine | Ala | A |
| allosoleucine | AIle | |
| Arginine | Arg | R |
| asparagine | Asn | N |
| aspartic acid | Asp | D |
| Cysteine | Cys | C |
| glutamic acid | Glu | E |
| Glutamine | Gln | Q |
| Glycine | Gly | G |
| Histidine | His | H |
| Isolelucine | Ile | I |
| Leucine | Leu | L |
| Lysine | Lys | K |
| phenylalanine | Phe | F |
| proline | Pro | P |
| pyroglutamic acid | pGlu | |
| Serine | Ser | S |
| Threonine | Thr | T |
| Tyrosine | Tyr | Y |
| Tryptophan | Trp | W |
| Valine | Val | V |

TABLE 2

Amino Acid Substitutions
Original Residue Exemplary Conservative Substitutions, others are known in the art

| | |
|---|---|
| Ala | Ser |
| Arg | Lys; Gln |
| Asn | Gln; His |
| Asp | Glu |
| Cys | Ser |
| Gln | Asn, Lys |
| Glu | Asp |
| Gly | Pro |
| His | Asn;Gln |
| Ile | Leu; Val |
| Leu | Ile; Val |
| Lys | Arg; Gln |
| Met | Leu; Ile |
| Phe | Met; Leu; Tyr |
| Ser | Thr |
| Thr | Ser |
| Trp | Tyr |
| Tyr | Trp; Phe |
| Val | Ile; Leu |

74. Substantial changes in function or immunological identity are made by selecting substitutions that are less conservative than those in Table 2, i.e., selecting residues that differ more significantly in their effect on maintaining (a) the structure of the polypeptide backbone in the area of the substitution, for example as a sheet or helical conformation, (b) the charge or hydrophobicity of the molecule at the target site or (c) the bulk of the side chain. The substitutions which in general are expected to produce the greatest changes in the protein properties will be those in which (a) a hydrophilic residue, e.g. seryl or threonyl, is substituted for (or by) a hydrophobic residue, e.g. leucyl, isoleucyl, phenylalanyl, valyl or alanyl; (b) a cysteine or proline is substituted for (or by) any other residue; (c) a residue having an electropositive side chain, e.g., lysyl, arginyl, or histidyl, is substituted for (or by) an electronegative residue, e.g., glutamyl or aspartyl; or (d) a residue having a bulky side chain, e.g., phenylalanine, is substituted for (or by) one not having a side chain, e.g., glycine, in this case, (e) by increasing the number of sites for sulfation and/or glycosylation.

75. For example, the replacement of one amino acid residue with another that is biologically and/or chemically similar is known to those skilled in the art as a conservative substitution. For example, a conservative substitution would be replacing one hydrophobic residue for another, or one polar residue for another. The substitutions include combinations such as, for example, Gly, Ala; Val, Ile, Leu; Asp, Glu; Asn, Gln; Ser, Thr; Lys, Arg; and Phe, Tyr. Such conservatively substituted variations of each explicitly disclosed sequence are included within the mosaic polypeptides provided herein.

76. Substitutional or deletional mutagenesis can be employed to insert sites for N-glycosylation (Asn-X-Thr/Ser) or O-glycosylation (Ser or Thr). Deletions of cysteine or other labile residues also may be desirable. Deletions or substitutions of potential proteolysis sites, e.g. Arg, is accomplished for example by deleting one of the basic residues or substituting one by glutaminyl or histidyl residues.

77. Certain post-translational derivatizations are the result of the action of recombinant host cells on the expressed polypeptide. Glutaminyl and asparaginyl residues are frequently post-translationally deamidated to the corresponding glutamyl and asparyl residues. Alternatively, these residues are deamidated under mildly acidic conditions. Other post-translational modifications include hydroxylation of proline and lysine, phosphorylation of hydroxyl groups of seryl or threonyl residues, methylation of the o-amino groups of lysine, arginine, and histidine side chains (T.E. Creighton, Proteins: Structure and Molecular Properties, W. H. Freeman & Co., San Francisco pp 79-86 [1983]), acetylation of the N-terminal amine and, in some instances, amidation of the C-terminal carboxyl.

78. It is understood that one way to define the variants and derivatives of the disclosed proteins herein is through defining the variants and derivatives in terms of homology/identity to specific known sequences. Specifically disclosed are variants of these and other proteins herein disclosed which have at least, 70% or 75% or 80% or 85% or 90% or 95% homology to the stated sequence. Those of skill in the art readily understand how to determine the homology of two proteins. For example, the homology can be calculated after aligning the two sequences so that the homology is at its highest level.

79. Another way of calculating homology can be performed by published algorithms. Optimal alignment of sequences for comparison may be conducted by the local homology algorithm of Smith and Waterman Adv. Appl. Math. 2: 482 (1981), by the homology alignment algorithm of Needleman and Wunsch, J. MoL Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, WI), or by inspection.

80. The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989.

81. It is understood that the description of conservative mutations and homology can be combined together in any combination, such as embodiments that have at least 70% homology to a particular sequence wherein the variants are conservative mutations.

82. As this specification discusses various proteins and protein sequences it is understood that the nucleic acids that can encode those protein sequences are also disclosed. This would include all degenerate sequences related to a specific protein sequence, i.e. all nucleic acids having a sequence that encodes one particular protein sequence as well as all nucleic acids, including degenerate nucleic acids, encoding the disclosed variants and derivatives of the protein sequences. Thus, while each particular nucleic acid sequence may not be written out herein, it is understood that each and every sequence is in fact disclosed and described herein through the disclosed protein sequence. It is also understood that while no amino acid sequence indicates what particular DNA sequence encodes that protein within an organism, where particular variants of a disclosed protein are disclosed herein, the known nucleic acid sequence that encodes that protein from which that protein arises are also known and herein disclosed and described.

83. It is understood that there are numerous amino acid and peptide analogs which can be incorporated into the disclosed compositions. For example, there are numerous D amino acids or amino acids which have a different functional substituent then the amino acids shown in Table 1 and Table 2. The opposite stereo isomers of naturally occurring peptides are disclosed, as well as the stereo isomers of peptide analogs. These amino acids can readily be incorporated into polypeptide chains by charging tRNA molecules with the amino acid of choice and engineering genetic constructs that utilize, for example, amber codons, to insert the analog amino acid into a peptide chain in a site-specific way.

84. Molecules can be produced that resemble peptides, but which are not connected via a natural peptide linkage. For example, linkages for amino acids or amino acid analogs can include CH₂NH—, —CH₂S—, —CH₂—CH₂ —, —CH═CH— (cis and trans), —COCH₂ —, —CH(OH)CH₂—, and --CHHzSO--(These and others can be found in Spatola, A. F. in Chemistry and Biochemistry of Amino Acids, Peptides, and Proteins, B. Weinstein, eds., Marcel Dekker, New York, p. 267 (1983); Spatola, A. F., Vega Data (March 1983), Vol. 1, Issue 3, Peptide Backbone Modifications (general review); Morley, Trends Pharm Sci (1980) pp. 463-468; Hudson, D. et al., Int J Pept Prot Res 14:177-185 (1979) (—CH₂NH—, CH₂CH₂—); Spatola et al. Life Sci 38:1243-1249 (1986) (—CH H₂—S); Hann J. Chem. Soc Perkin Trans. I307-314 (1982) (—CH—CH—, cis and trans); Almquist et al. J. Med. Chem. 23:1392-1398 (1980) (—COCH₂—); Jennings-White et al. Tetrahedron Lett 23:2533 (1982) (—COCH₂—). Szelke et al. European Appln, EP 45665 CA (1982): 97:39405 (1982) (—CH(OH)CH₂—), Holladay et al. Tetrahedron. Lett 24:4401-4404 (1983) (—C(OH)CH₂—); and Hruby Life Sci 31:189-199 (1982) (—CH₂—S—), each of which is incorporated herein by reference. A particularly preferred non-peptide linkage is —CH₂NH—. It is understood that peptide analogs can have more than one atom between the bond atoms, such as b-alanine, g-aminobutyric acid, and the like.

85. Amino acid analogs and analogs and peptide analogs often have enhanced or desirable properties, such as, more economical production, greater chemical stability, enhanced pharmacological properties (half-life, absorption, potency, efficacy, etc.), altered specificity (e.g., a broad-spectrum of biological activities), reduced antigenicity, and others.

86. D-amino acids can be used to generate more stable peptides, because D amino acids are not recognized by peptidases and such. Systematic substitution of one or more amino acids of a consensus sequence with a D-amino acid of the same type (e.g., D-lysine in place of L-lysine) can be used to generate more stable peptides. Cysteine residues can be used to cyclize or attach two or more peptides together. This can be beneficial to constrain peptides into particular conformations.

### 5. Pharmaceutical Carriers/Delivery of Pharmaceutical Products

87. As described above, the compositions can also be administered in vivo in a pharmaceutically acceptable carrier. By “pharmaceutically acceptable” is meant a material that is not biologically or otherwise undesirable, i.e., the material may be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

88. The compositions (i.e., the engineered AAV vectors disclosed herein) may be administered orally, parenterally (e.g., intravenously), by intramuscular injection, by intraperitoneal injection, transdermally, by cerebrospinal fluid injection, extracorporeally, topically or the like, including topical intranasal administration or administration by inhalant. As used herein, “topical intranasal administration” means delivery of the compositions into the nose and nasal passages through one or both of the nares and can comprise delivery by a spraying mechanism or droplet mechanism, or through aerosolization of the nucleic acid or vector. Administration of the compositions by inhalant can be through the nose or mouth via delivery by a spraying or droplet mechanism. Delivery can also be directly to any area of the respiratory system (e.g., lungs) via intubation. The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

89. Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

90. The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K.D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis have been reviewed (Brown and Greene, DNA and CellBiology 10:6, 399-409 (1991)).

### A) Pharmaceutically Acceptable Carriers

91. The compositions, including antibodies, can be used therapeutically in combination with a pharmaceutically acceptable carrier.

92. Suitable carriers and their formulations are described in Remington: The Science and Practice of Pharmacy (19th ed.) ed. A.R. Gennaro, Mack Publishing Company, Easton, PA 1995. Typically, an appropriate amount of a pharmaceutically-acceptable salt is used in the formulation to render the formulation isotonic. Examples of the pharmaceutically-acceptable carrier include, but are not limited to, saline, Ringer’s solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

93. Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

94. Pharmaceutical compositions may include carriers, thickeners, diluents, buffers, preservatives, surface active agents and the like in addition to the molecule of choice. Pharmaceutical compositions may also include one or more active ingredients such as antimicrobial agents, antiinflammatory agents, anesthetics, and the like.

95. The pharmaceutical composition may be administered in a number of ways depending on whether local or systemic treatment is desired, and on the area to be treated. Administration may be topically (including ophthalmically, vaginally, rectally, intranasally), orally, by inhalation, or parenterally, for example by intravenous drip, cerebrospinal fluid injection, subcutaneous injection, intraperitoneal injection, or intramuscular injection. The disclosed engineered viral vectors can be administered intravenously, intraperitoneally, intramuscularly, subcutaneously, intracavity, or transdermally.

96. Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer’s dextrose, dextrose and sodium chloride, lactated Ringer’s, or fixed oils. Intravenous vehicles include fluid and nutrient replenishers, electrolyte replenishers (such as those based on Ringer’s dextrose), and the like. Preservatives and other additives may also be present such as, for example, antimicrobials, anti-oxidants, chelating agents, and inert gases and the like.

97. Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

98. Compositions for oral administration include powders or granules, suspensions or solutions in water or non-aqueous media, capsules, sachets, or tablets. Thickeners, flavorings, diluents, emulsifiers, dispersing aids or binders may be desirable.

99. Some of the compositions may potentially be administered as a pharmaceutically acceptable acid- or base- addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

### B) Therapeutic Uses

100. Effective dosages and schedules for administering the compositions may be determined empirically, and making such determinations is within the skill in the art. The dosage ranges for the administration of the compositions are those large enough to produce the desired effect in which the symptoms of the disorder are affected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any counterindications. Dosage can vary, and can be administered in one or more dose administrations daily, for one or several days. Guidance can be found in the literature for appropriate dosages for given classes of pharmaceutical products. For example, guidance in selecting appropriate doses for antibodies can be found in the literature on therapeutic uses of antibodies, e.g., Handbook of Monoclonal Antibodies, Ferrone et al., eds., Noges Publications, Park Ridge, N.J., (1985) ch. 22 and pp. 303-357; Smith et al., Antibodies in Human Diagnosis and Therapy, Haber et al., eds., Raven Press, New York (1977) pp. 365-389. A typical daily dosage of the antibody used alone might range from about 1 µg/kg to up to 100 mg/kg of body weight or more per day, depending on the factors mentioned above.

### C. Methods of Treating Neurological Disease

101. In one aspect, disclosed herein are methods of treating, inhibiting, decreasing, reducing, ameliorating, and/or preventing a neurological disease (such as, for example, Alzheimer’s disease, Parkinson’s disease, Multiple Systems Atrophy (MSA), Lysosomal Storage Disease (LSD), and/or muscular dystrophy) in a subject comprising administering to the subject a therapeutic agent encoded by the engineered AAV vector disclosed herein. The therapeutic agent can be any therapeutic gene whose expression can rescue a loss of function including, but not limited to of GLB1 (which encodes b-galactosidase for the treatment of GM1 Ganliosidosis), Niemann-Pick C1 (NPC1)(for the treatment of Neimann-Pick disease), Apolipoprotein E (APOEXfor the treatment of Alzheimer’s disease), GD3 synthase, huntingtin (Htt)(for the treatment of Huntington’s disease), interleukin (IL)-10 (IL-10)for the treatment of disorders including, but not limited to Multiple Sclerosis, Traumatic Brain Injury. Amyotrophic lateral sclerosis, Alzheimer’s Disease, and Parkinson’s Disease), Myelin Oligodendrocyte Glycoprotein (MOG)(for the treatment of narcolepsy), mitogen-activated protein kinase 8 interacting protein 3 (MAPKA8IP3)for the treatment of spastic diplegia, intellectual disability, cerebral atrophy and/or corpus callosum hypoplasia), survival motor neuron (SMN) 1 (SMN1) and/or SMN2 (for the treatment of spinal muscular atrophy (SMA)), Cas9, β-Glucocerebrosidase (GBA)(for the treatment of Gaucher disease and/or lysosomal storage disease (LSD)), Sphingomyelin phosphodiesterase 1 (SMPD1) (for the treatment of Niemann-Pick disease, Parkinson’s disease, and/or lysosomal storage disease), beta-hexosaminidase A (HEXA)(for the treatment of Tay-Sachs disease and/or lysosomal storage disease), nerve growth factor (NGF)(for treatment of pain and age-related neurodegenerative diseases), brain-derived neurotrophic factor (BDNF)(for the treatment of age-related and pathological neurological disorders), neurotrophin (NT) 3 (NT-3)(for the treatment of Autism spectrum diseases, Alzheimer’s disease, Huntington’s disease, and/or Parkinson’s disease), NT-⅘ (for the treatment of Alzheimer’s disease, Huntington’s disease, and/or Parkinson’s disease), NT-6 (for the treatment of Alzheimer’s disease, Huntington’s disease, and/or Parkinson’s disease), Glial Cell Derived Neurotrophic Factor (GDNF)(for the treatment of Alzheimer’s disease and/or Multiple Systems Atrophy (MSA)), Ciliary Neurotrophic Factor (CNTF), Leukemia inhibitory factor (LIF). Insulin-like growth factor (IGF) 1 (IGF-1), b-fibroblast growth factor (FGF), neurturin (for the treatment of Parkinson’s disease), persephin (for the treatment of Parkinson’s disease), artemin (for the treatment of Parkinson’s disease), transforming growth factor (TGF) alpha (TGFα), TGFβ, IGF-2, platelet derived growth factor (PDGF), epidermal growth factor (EGF), cardiotropin, vascular endothelial growth factor (VEGF), Sonic hedgehog (SHH)(for treatment of Holoprosencephaly), bone morphogenic proteins (BMP), FGF20, Vasoactive Intestinal Peptide (VIP), pleiotrophin (PTN), Aromatic L-amino Acid Decarboxylase (AADC), TH, 5-hydroxytryptamine (5HT), and hepatocyte growth factor (HGF). As noted above, the therapeutic agent can also comprise a microRNA including but not limited to miRNA-222, miRNA-7, and miRNA-132.

102. Also disclosed are methods of treating, inhibiting, decreasing, reducing, ameliorating, and/or preventing a neurological disease of any preceding aspect, wherein the AAV vector is administered systemically (such as, for example, intravenously, including but not limited to, i.v. injection or i.v. drip; retro-orbitally); or via cerebrospinal fluid injection).

103. The disclosed AAV vectors can encode any peptide, protein, antibody, or nucleic acid appropriate for the treatment of the neurological disease or condition. Examples of such nucleic acids, peptides, proteins, antibodies known to those of skill in the art.

### D. Examples

104. The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in °C or is at ambient temperature, and pressure is at or near atmospheric.

### 1. Example 1: Generation of LC.VI Capsid

105. Recombinant AAV vectors have become an attractive gene delivery vehicle because they can transduce both dividing and post-mitotic tissues with low immunogenicity and long-lasting transgene expression. AAV2-mediated gene transfer to tissues such as liver, muscle, retina and central nervous system has been reported. Later, new non-human serotypes such as AAV8 were isolated and identified through PCR-based screening of primate tissues, and was shown to transduce neurons better than that of AAV2. Furthermore, comparison among these new primate serotypes revealed widespread neuronal transduction following infusion of cy5, rh20 and rh39, to a level greater than that of AAV8. Based on this study, a series of hybrid recombinant capsids, including Rec2 capsid, were generated by structural domain exchange or shuffling among fragments from cy5, rh20 and rh39, in hope that their tropisms could render the novel hybrid serotypes efficiently target retina. In vivo and in vitro evaluations showed the transduction efficacy of Rec2 capsid no better than that of AAV2 or AAV5. However, surprisingly, Rec2 serotype exhibits widespread transduction in both brown and white adipose tissue superior to the naturally occurring serotypes tested including AAV1, AAV8, and AAV9 serotypes. Since the first publication of Rec2-mediated gene transfer to adipose tissue, Rec2 serotype vectors have been applied in basic and translation research. In addition, Rec2 can also transduce liver effectively via intravenous injection in order to improve the transduction efficacy to adipose tissue and meanwhile reduce transduction to liver, several variants of Rec2 capsids were created by point mutations or insertional mutation.

Capsid (cap) gene in AAV2 genome via an alternative splicing and initiation encodes structural viral proteins (VPs), including VP1, VP2 and VP3, thus all three VP proteins share identical carboxyl-terminal amino acids. VP1, VP2, and VP3 make up an AAV capsid with each subunit a molar ratio of 1:1:10 to form an icosahedron structure. AAV tropism dictates cell entry. AAV2 makes a cell entry by using membrane-associated heparan sulfate proteoglycan (HSPG) as its primary receptor. Other candidates have also been reported to participate AAV attachment. The atomic structure of AAV2 capsid has been determined at a resolution of 3.0 micron. Based on the computer modeling, some genetic capsids modifications can be tolerated for AAV2 serotype. For example, work suggested potential heparin binding loci that include a cluster between position 561 and 591, and was re-confirmed by recent comprehensive AAV capsid fitness landscape study. Further studies identify residues R585 and R588 as two of primary amino acid residues responsible for HSPG binding. Several studies have reported genetic modification focused on the N587 and I588 surrounding residues for vector re-targeting. Thus, it was decided to make mutations on Rec2 capsid in the region equivalent to the region surrounding R585 and R588 of AAV2 capsid in order to screen new serotype capsids improved adipose tropism. Recently, genetic modifications were expanded beyond the R585~R588 region (Table 3). Initial in vivo screening showed all variants lower transduction efficiency to adipose tissue or liver compared to the native Rec2 capsid. For example, the variant 1, namely LC.V1 capsid lost transduction efficiency to adipose tissue by 76% via intra-adipose injection and by 83% via intraperitoneal injection compared to native Rec2 capsid. Moreover, LC.V1 showed 50% lower transduction efficiency to liver compared to native Rec2 capsid. These variants were deemed failure of the original intention to generate adipose-specific AAV serotypes, and later were screened for transduction to other tissues. Surprisingly, the variant 1, namely LC.V1 capsid displayed high transduction to neurons and widespread transgene expression in mouse brain (FIG. 1) and rat brain (FIGS. 2, 3). Specifically, LC.V1 vector expressing green fluorescent protein (GFP) was injected to the striatum of C57BL/6 mouse unilaterally at the dose of 1 × 10⁹ viral particles in 1 µL. The transduction range was ~3 mm which is approximately 15% of the mouse brain (FIG. 1). LC.V1 vector displayed similar widespread transduction in Sprague-Dawley rat brain via Convection-enhanced Delivery (CED) to striatum unilaterally (1.8 × 10¹¹ viral particles in 15 µL). Surprisingly, robust transduction of the rat brain was observed in many brain structures including striatum (targeting structure), cortex, septum, hippocampus, thalamus, subthalamic nucleus, substantia nigra, etc. (FIG. 2, FIG. 3). The spread of the vector from the target structure extended to over 3.5 mm in the anterior and 5 mm in the posterior direction. The LC.V1 vector appeared to transduce predominantly neurons at very high efficiency, and to be transported in a retrograde direction.

TABLE 3

Genetic modification of Rec2 capsid

| | | |
|---|---|---|
| Variants | Mutations | In vivo testing performed |
| LC.V1 | Q588P, Q589L, Q594L | Mouse adipose tissue, liver Mouse brain, rat brain |
| LC.V2 | N540I, N549I, D559V | Mouse adipose tissue, liver |
| LC.V3 | CSWKYWFGEC (SEQ ID NO: 2) insertion at 585 | Unable to package |
| LC.V4 | Replacement of ATEEYGTVAD (575-585) (SEQ ID NO: 3) by CSWKYWFGEC at (575-585) | Mouse adipose tissue, liver |
| LC.V5 | F503Y | Mouse adipose tissue, liver |
| LC.V6 | A458T, Q461S, N471S | Mouse adipose tissue, liver |

### 2. Example 2: Construction of LC.V1 Capsid and GFP Reporter Vector Packaging

106. Rec2 capsid was mutagenized at Q588P, Q589L and Q594L through GeneArt site-directed mutagenesis Plus kit (A14604, Invitrogen). Rec2 capsid sequence is based on published data. Primers for mutagenesis was synthesized by Integrated DNA Technologies (IDT) and listed as following, VP3_1763-FW/ CAGATAACTTGCAGCCGCTAAACACGGCT CCTCTAATTGGAACTGTCAA (SEQ ID NO: 4), VP3_1763RV/ TTGACAGTTCCAATTAG AGGAGCCGTGTTTAGCGGCTGCAAGTTATCTG (SEQ ID NO: 5)

107. Mutagenesis was set up and carried out according to the kit instruction, and the nucleotide replacement at each site was confirmed by sequencing at the Core Facility of The Ohio State University, Comprehensive Cancer Center. rAAV vector backbone contains CBA (hybrid cytomegalovirus-chicken β-actin) promoter, woodchuck hepatitis virus posttranscriptional regulatory element (WPRE) and bovine growth hormone polyadenylation signal flanked by AAV2-inverted terminal repeats. GFP was cloned into polylinker sites of rAAV expression plasmid. Plasmids used for viral packaging were all prepared using EndoFree plasmid Maxi and Mega Kit (Qiagen). Human embryonic kidney 293 cells were co-transfected with three plasmids-rAAV cis-plasmid containing GFP as reporter gene, AAV helper plasmid encoding rep and cap (Rec2) genes and adenoviral helper pF Δ6-using standard CaPO₄ transfection. rAAV was purified from the cell lysate by ultracentrifugation through an iodixanol density gradient (OptiPrep Density Gradient Medium, D1556, Sigma). rAAV was titered by quantitative PCR using Step OnePlus Real-Time PCR System (Applied Biosystems) with the Power SYBR Green PCR Master Mix (Applied Biosystems#A25742).

108. One of the most important characteristics of gene therapy vectors is their efficiency to transduce the target cells. In gene therapy of the central nervous system (CNS) scientists are constantly looking for “best performers” that would efficiently spread within the entire brain and transduce the greatest number of neurons. This would warrant the best treatment outcomes for many neurodegenerative diseases affecting large areas of the brain (Alzheimer’s, Parkinson’s, lysosomal storage disorders). We have evaluated the efficiency of transduction of LC.V1 vector delivered into the non-human primate brain via Convection-enhanced Delivery (CED). Briefly, we injected 124 µl of LC.V1 into the left monkey thalamus (rate of infusion 3 µl/min) and 59 µl into the right midbrain (the ventral tegmental area - VTA + substantia nigra) by means of MRI-guided CED. Animals were sedated and then positioned in a stereotactic system with MR-compatible, skull-mounted, temporary cannula guides placed over each hemisphere. After a T1-weighted planning scan (Siemens 3.0 T Trio MR unit) to set the trajectory, a ceramic custom-designed fused silica reflux-resistant cannula with a 3-mm stepped tip was used for the infusion. To visualize the infusate distribution during MRI, Prohance (2 mmol/1 chelated Gadolinium) was added to the virus. Serial MRIs were acquired to monitor the infusate distribution within each target site and to provide real-time feedback to the surgical team. Animals were euthanized after 3 weeks and the brains were processed for immunohistochemical staining to assess the efficiency of distribution and transduction. Double fluorescence staining against the transgene, GFP, and neuronal marker, NeuN, was used to determine the percentage of transduced neurons (efficiency of transduction).

109. The results from the transduction of the non-human primate brain with LC.V1 showed that this vector is extremely efficient in the distribution and efficiency of transduction of large areas of the brain. Specifically, the thalamus demonstrated near-complete coverage based on transgene expression (GFP) at the site of injection of only 124 µl of the LC.V1 vector (FIGS. 4A-C).The average neuronal transduction efficiency from 4 thalamic nuclei (Ventral Anterior Lateral, Ventral Medial Lateral, Lateral Dorsal, and Medial Dorsal) was 67% (FIG. 5I) as measured by GFP expression. LC.V1 is transported retrogradely from the site of injection (thalamus) to cortical regions where it transduces pyramidal neurons of prefrontal cortex and frontal cortex layer V (FIGS. 4D-K and FIGS. 5A-C). This is significant as it supports the use of LC.v1 for treatment of disease affecting the prefrontal cortex. The neuronal transduction efficiency within the prefrontal cortex was 50% (FIG. 5I) based on the average of 8 cortical regions, 4 lateral and medial, from 3 separate sections of brain tissue, all within Area 9 of prefrontal cortex. LC.V1 is transported retrogradely from the site of injection (thalamus) to the hippocampus and transduces neurons of the subiculum (FIGS. 5D-H). The average neuronal transduction efficiency within this structure is 53% (FIG. 5I). LC.V1 is transported anterogradely from the site of injection (midbrain: VTA and substantia nigra) to the striatum (both caudate nucleus and putamen) where numerous GFP-positive fibers can be seen (FIGS. 6A-D). Distribution of LC.V1 within the brain parenchyma delivered via CED can be monitored by real-time MRI imaging. By including the MRI contrast agent, ProHance (gadoteridol), one can track the convective movement of LC.V 1 viral particles by continuous monitoring of distribution of ProHance. MRI scans perfectly correlate with histological (immunofluorescence staining - IF) brain sections showing expression of the transgene, GFP (FIGS. 7A-F). The data shown herein also support the observation that LC.V1 is bidirectional showing both retrograde and anterograde transport. Also, we were able to monitor the vector distribution in real time. This is significant as an administering physician can monitor the vector administration and stop or adjust infusion when primary target is filled or being delivered to the correct location.

110. Next, we wanted to assess non-parenchymal routes of delivery like systemic and CSF by evaluating the level of transgene transduction (via GFP expression) at different brain levels from the anterior-posterior axis (FIG. 8), peripheral organs (FIG. 9), and in hippocampus and dentate gyrus from representative animals (FIG. 10). We observed that LC.V1 was globally and evenly distributed regardless of non-parenchymal routes of delivery. Positive GFP signal was also found along all cortical lobes (prefrontal, frontal, temporal, parietal, and occipital) when lateral tail vein injection, CSF, or lateral ventricle injection was used. Next, we assayed both the neuronal and astrocytic tropism. Here, LC.V1 appeared to transduce neurons (FIG. 11) and interneurons in all the cortical and subcortical areas (pyramidal cell, medium spiny neurons, dopaminergic neurons, Purkinje etc). We noted that while several rAAV (specifically AAV9) have been shown to cross the blood brain barrier in neonatal mouse central nervous systems, the same claim has not been shown in adult subjects. When compared to AAV9.HR, we injected similar doses (4.0E+12 vg vs. 4.18E+12 vg), LC.V1 performed at better distribution and higher levels of expression. When compared to AAV-PHP.B, we injected higher dose (E+12 vg vs. 4.18E+12 vg), LC.V1 performed at similar/better distribution and levels of expression.

111. To perform these experiments, animals (C57BL/6 mice) received either systemic injection throughout right retro-orbital sinus (RO) or Tail vein (TV); or CSF injection through the left lateral ventricle delivery (LV). Each animals received 50 µL systemically or 25 µL into the CSF of AAV:LC.V1 at a titer of 8.36E+13 vg/mL (lot# CS1851). Injection into the left lateral ventricle (also known as intraventricular delivery) was achieved using a custom- made fused silica 1 mm stepped canula. Vector delivery was stereotactically guided using the following coordinates from mouse brain atlas. Anterior - Posterior: -0.3 mm; Medial - Lateral: +0.9 mm; Dorsal - Ventral: -1.8 mm.

### E. Sequences

### SEQ ID NO: 1 Amino Acid Sequence of Rec2 Capsid Protein

MAADGYLPDWLEDNLSEGIREWWDLKPGAPKPKANQQKQDDGRGLVLPGY
50KYLGPFNGLDKGEPVNAADAAALEHDKAYDQQLKAGDNPYLRYNHAD
AEF 100QERLQEDTSFGGNLGRAVFQAKKRVLEPLGLVEEGAKTAPGKK
RPVEPSP 150QRSPDSSTGIGKTGQQPAKKRLNFGQTGDSESVPDPQPI
GEPPAGPSGLG 200SGTMAAGGGAPMADNNEGADGVGSSSGNWHCDSTW
LGDRVITTSTRTWAL 250PTYNNHLYKQISNGTSGGSTNDNTYFGYSTP
WGYFDFNRFHCHFSPRDWQ 300RLINNNWGFRPKRLNFKLFNIQVKEVT
QNEGTKTIANNLTSTIQVFTDSE 350YQLPYVLGSAHQGCLPPFPADVF
MIPQYGYLTLNNGSQAVGRSSFYCLEY 400FPSQMLRTGNNFEFSYQFE
DVPFHSSYAHSQSLDRLMNPLIDQYLYYLSR 450TQSTGGTAGTQQLLF
SQAGPNNMSAQAKNWLPGPCYRQQRVSTTTGQNNN 500SNFAWTAGTKY
HLNGRNSLANPGIAMATHKDDEERFFPSNGILIFGKQNA 550ARDNADY
SDVMLTSEEEIKTTNPVATEEYGIVADNLQQQNTAPQIGTVNS 600QGA
LPGMVWQNRDVYLQGPIWAKIPHTDGNFHPSPLMGGFGLKHPPPQIL 65
0IKNTPVPADPPTTFNQSKLNSFITQYSTGQVSVEIEWELQKENSKRWNP
E 700IQYTSNYYKSTSVDFAVNTEGVYSEPRPIGTRYLTRNL 738

### SEQ ID NO: 6 Amino Acid Sequence of LC.V1 Capsid

MAADGYLPDWLEDNLSEGIREWWDLKPGAPKPKANQQKQDDGRGLVLPGY
50KYLGPFNGLDKGEPVNAADAAALEHDKAYDQQLKAGDNPYLRYNHAD
AEF 100QERLQEDTSFGGNLGRAVFQAKKRVLEPLGLVEEGAKTAPGKK
RPVEPSP 150QRSPDSSTGIGKTGQQPAKKRLNFGQTGDSESVPDPQPI
GEPPAGPSGLG 200SGTMAAGGGAPMADNNEGADGVGSSSGNWHCDSTW
LGDRVITTSTRTWAL 250PTYNNHLYKQISNGTSGGSTNDNTYFGYSTP
WGYFDFNRFHCHFSPRDWQ 300RLINNNWGFRPKRLNFKLFNIQVKEVT
QNEGTKTIANNLTSTIQVFTDSE 350YQLPYVLGSAHQGCLPPFPADVF
MIPQYGYLTLNNGSQAVGRSSFYCLEY 400FPSQMLRTGNNFEFSYQFE
DVPFHSSYAHSQSLDRLMNPLIDQYLYYLSR 450TQSTGGTAGTQQLLF
SQAGPNNMSAQAKNWLPGPCYRQQRVSTTTGQNNN 500SNFAWTAGTKY
HLNGRNSLANPGIAMATHKDDEERFFPSNGILIFGKQNA 550ARDNADY
SDVMLTSEEEIKTTNPVATEEYGIVADNLQPLNTAPLIGTVNS 600QGA
LPGMVWQNRDVYLQGPIWAKIPHTDGNFHPSPLMGGFGLKHPPPQIL 65
0IKNTPVPADPPTTFNQSKLNSFITQYSTGQVSVEIEWELQKENSKRWNP
E 700IQYTSNYYKSTSVDFAVNTEGVYSEPRPIGTRYLTRNL 738

### F. References

Becerra SP, Koczot F, Fabisch P, Rose JA. 1988. Synthesis of adeno-associated virus structural proteins requires both alternative mRNA splicing and alternative initiations from a single transcript. J Virol 62: 2745-54
Buning H, Huber A, Zhang L, Meumann N, Hacker U. 2015. Engineering the AAV capsid to optimize vector-host-interactions.Curr Opin Pharmacol 24: 94-104
Charbel Issa P, De Silva SR, Lipinski DM, Singh MS, Mouravlev A, You Q, Barnard AR, Hankins MW, During MJ, Maclaren RE. 2013. Assessment of tropism and effectiveness of new primate-derived hybrid recombinant AAV serotypes in the mouse and primate retina. PLoS One 8: e60361
Deverman BE, Pravdo PL, Simpson BP, Kumar SR, Chan KY, Banerjee A, Wu WL, Yang B, Huber N, Pasca SP, Gradinaru V (2016) Cre-dependent selection yields AAV variants for widespread gene transfer to the adult brain. Nat Biotechnol 34:204-209
Duan D, Li Q, Kao AW, Yue Y, Pessin JE, Engelhardt JF. 1999. Dynamin is required for recombinant adeno-associated virus type 2 infection. J Virol 73: 10371-6
During MJ, Liu X, Huang W, Magee D, Slater A, McMurphy T, Wang C, Cao L. 2015. Adipose VEGF Links the White-to-Brown Fat Switch With Environmental, Genetic, and Pharmacological Stimuli in Male Mice. Endocrinology 156: 2059-73
Gao G, Vandenberghe LH, Wilson JM. 2005. New recombinant serotypes of AAV vectors. Curr Gene Ther 5: 285-97
Gao GP, Alvira MR, Wang L, Calcedo R, Johnston J, Wilson JM. 2002. Novel adeno-associated viruses from rhesus monkeys as vectors for human gene therapy. Proc Natl Acad Sci U S A 99: 11854-9
Girod A, Ried M, Wobus C, Lahm H, Leike K, Kleinschmidt J, Deleage G, Hallek M. 1999. Genetic capsid modifications allow efficient re-targeting of adeno-associated virus type 2. Nat Med 5: 1052-6
Huang W, Liu X, Queen NJ, Cao L. 2017. Targeting Visceral Fat by Intraperitoneal Delivery of Novel AAV Serotype Vector Restricting Off-Target Transduction in Liver. Mol Ther Methods Clin Dev 6: 68-78
Huang W, McMurphy T, Liu X, Wang C, Cao L. 2016. Genetic Manipulation of Brown Fat Via Oral Administration of an Engineered Recombinant Adeno-associated Viral Serotype Vector. Mol Ther 24: 1062-9
Huang W, Queen NJ, Cao L. 2019. rAAV-Mediated Gene Delivery to Adipose Tissue. Methods Mol Biol 1950: 389-405
Huang W, Queen NJ, McMurphy TB, Ali S, Cao L. 2019. Adipose PTEN regulates adult adipose tissue homeostasis and redistribution via a PTEN-leptin-sympathetic loop. Mol Metab 30: 48-60
KD Foust, E Nurre, CL Montgomery. A Hernandez, CM Chan, BK Kaspar (2009). Intravascular AAV9 preferentially targets neonatal neurons and adult astrocytes. Nat Biotechnol, 27 (2009), pp. 59-65
Kern A, Schmidt K, Leder C, Muller OJ, Wobus CE, Bettinger K, Von der Lieth CW, King JA, Kleinschmidt JA. 2003. Identification of a heparin-binding motif on adeno-associated virus type 2 capsids. J Virol 77: 11072-81
Klein RL, Dayton RD, Leidenheimer NJ, Jansen K, Golde TE, Zweig RM. 2006. Efficient neuronal gene transfer with AAV8 leads to neurotoxic levels of tau or green fluorescent proteins. Mol Ther 13: 517-27
Lawlor PA, Bland RJ, Mouravlev A, Young D, During MJ. 2009. Efficient gene delivery and selective transduction of glial cells in the mammalian brain by AAV serotypes isolated from nonhuman primates. Mol Ther 17: 1692-702
Liu X, Magee D, Wang C, McMurphy T, Slater A, During M. Cao L. 2014. Adipose tissue insulin receptor knockdown via a new primate-derived hybrid recombinant AAV serotype.Mol Ther Methods Clin Dev 1
Lluis Samaranch, Ernesto A Salegio, Waldy San Sebastian, Adrian P Kells, John R Bringas, John Forsayeth, Krystof S Bankiewicz (2013) Strong cortical and spinal cord transduction after AAV7 and AAV9 delivery into the cerebrospinal fluid of nonhuman primates. Hum Gene Ther. 2013 May;24(5):526-32
McMurphy TB, Huang W, Xiao R, Liu X, Dhurandhar NV, Cao L. 2017. Hepatic Expression of Adenovirus 36 E4ORF1 Improves Glycemic Control and Promotes Glucose Metabolism Through AKT Activation. Diabetes 66: 358-71
Mingozzi F, High KA. 2011. Therapeutic in vivo gene transfer for genetic disease using AAV: progress and challenges. Nat Rev Genet 12: 341-55
Ng R, Hussain NA, Zhang Q, Chang C, Li H, Fu Y, Cao L, Han W, Stunkel W, Xu F. 2017. miRNA-32 Drives Brown Fat Thermogenesis and Trans-activates Subcutaneous White Fat Browning in Mice. Cell Rep 19: 1229-46
Nicklin SA, Buening H, Dishart KL, de Alwis M, Girod A, Hacker U, Thrasher AJ, Ali RR, Hallek M, Baker AH. 2001. Efficient and selective AAV2-mediated gene transfer directed to human vascular endothelial cells. Mol Ther 4: 174-81
Ogden PJ, Kelsic ED, Sinai S, Church GM. 2019. Comprehensive AAV capsid fitness landscape reveals a viral gene and enables machine-guided design. Science 366: 1139-43
Opie SR, Warrington KH, Jr., Agbandje-McKenna M, Zolotukhin S, Muzyczka N. 2003. Identification of amino acid residues in the capsid proteins of adeno-associated virus type 2 that contribute to heparan sulfate proteoglycan binding. J Virol 77: 6995-7006
Perabo L, Goldnau D, White K, Endell J, Boucas J, Humme S, Work LM, Janicki H, Hallek M, Baker AH, Buning H. 2006. Heparan sulfate proteoglycan binding properties of adeno-associated virus retargeting mutants and consequences for their in vivo tropism. J Virol 80: 7265-9
Pillay S, Meyer NL, Puschnik AS, Davulcu O, Diep J, Ishikawa Y, Jae LT, Wosen JE, Nagamine CM, Chapman MS, Carette JE. 2016. An essential receptor for adeno-associated virus infection. Nature 530: 108-12
Qing K, Mah C, Hansen J, Zhou S, Dwarki V, Srivastava A. 1999. Human fibroblast growth factor receptor 1 is a co-receptor for infection by adeno-associated virus 2. Nat Med 5: 71-7
Rabinowitz JE, Samulski J. 1998. Adeno-associated virus expression systems for gene transfer. Curr Opin Biotechnol 9: 470-5
Rabinowitz JE, Xiao W, Samulski RJ. 1999. Insertional mutagenesis of AAV2 capsid and the production of recombinant virus. Virology 265: 274-85
Ried MU, Girod A, Leike K, Buning H, Hallek M. 2002. Adeno-associated virus capsids displaying immunoglobulin-binding domains permit antibody-mediated vector retargeting to specific cell surface receptors. J Virol 76: 4559-66
Samaranch L, Salegio EA, San SW, Kells AP, Foust KD, Bringas JR, Lamarre C, Forsayeth J, Kaspar BK, Bankiewicz KS (2012) Adeno-associated virus serotype 9 transduction in the central nervous system of nonhuman primates. Hum Gene Ther 23:382-389.
Summerford C, Bartlett JS, Samulski RJ. 1999. AlphaVbeta5 integrin: a co-receptor for adeno-associated virus type 2 infection. Nat Med 5: 78-82
Summerford C, Samulski RJ. 1998. Membrane-associated heparan sulfate proteoglycan is a receptor for adeno-associated virus type 2 virions. J Virol 72: 1438-45
Taymans JM, Vandenberghe LH, Haute CV, Thiry I, Deroose CM, Mortelmans L, Wilson JM, Debyser Z, Baekelandt V. 2007. Comparative analysis of adeno-associated viral vector serotypes 1, 2, 5, 7, and 8 in mouse brain. Hum Gene Ther 18: 195-206
Uhrig S, Coutelle O, Wiehe T, Perabo L, Hallek M, Buning H. 2012. Successful target cell transduction of capsid-engineered rAAV vectors requires clathrin-dependent endocytosis. Gene Ther 19: 210-8
Wang D, Li SY, Gessler DJ, Xie J, Zhong L, Tran K, Vliet KV, Ren LZ, Su Q, He R, Goetzmann JE, Flotte TR, Agbandje-McKenna M, Gao GP (2018) A rationally engineered capsid variant of AAV9 for systemic CNS-directed and peripheral tissue-detargeted gene delivery in neonates. Mol Ther-Methods Clin Dev 9:234-246.
Wu P, Xiao W, Conlon T, Hughes J, Agbandje-McKenna M, Ferkol T, Flotte T, Muzyczka N. 2000. Mutational analysis of the adeno-associated virus type 2 (AAV2) capsid gene and construction of AAV2 vectors with altered tropism. J Virol 74: 8635-47
Xiao R, Mansour AG, Huang W, Chrislip LA, Wilkins RK, Queen NJ, Youssef Y, Mao HC, Caligiuri MA, Cao L. 2019. Adipocytes: A Novel Target for IL-15/IL-15Ralpha Cancer Gene Therapy. Mol Ther 27: 922-32
Xie L, Zhang Y, Gunasekar SK, Mishra A, Cao L, Sah R. 2017. Induction of adipose and hepatic SWELL1 expression is required for maintaining systemic insulin-sensitivity in obesity. Channels (Austin) 11: 673-7
Xie Q, Bu W, Bhatia S, Hare J, Somasundaram T, Azzi A, Chapman MS. 2002. The atomic structure of adeno-associated virus (AAV-2), a vector for human gene therapy. Proc Natl Acad Sci U S A 99: 10405-10
Yang Q, Mamounas M, Yu G, Kennedy S, Leaker B, Merson J, Wong-Staal F, Yu M, Barber JR. 1998. Development of novel cell surface CD34-targeted recombinant adenoassociated virus vectors for gene therapy. Hum Gene Ther 9: 1929-37
Zhang H, Yang B, Mu X, Ahmed SS, Su Q, He R, Wang HY, Mueller C, Sena-Esteves M, Brown R, Xu ZS, Gao GP (2011) Several rAAV vectors efficiently cross the blood-brain barrier and transduce neurons and astrocytes in the neonatal mouse central nervous system. Mol Ther 19:1440-1448
Zhang R, Xu G, Cao L, Sun Z, He Y, Cui M, Sun Y, Li S, Li H, Qin L, Hu M, Yuan Z, Rao Z, Ding W, Rao Z, Lou Z. 2019. Divergent engagements between adeno-associated viruses with their cellular receptor AAVR. Nat Commun 10: 3760
Zhang Y, Xie L, Gunasekar SK, Tong D, Mishra A, Gibson WJ, Wang C, Fidler T, Marthaler B, Klingelhutz A, Abel ED, Samuel I, Smith JK, Cao L, Sah R. 2017. SWELL1 is a regulator of adipocyte size, insulin signalling and glucose homeostasis. Nat Cell Biol 19: 504-17
Zhu Y, Gao Y, Tao C, Shao M, Zhao S. Huang W, Yao T, Johnson JA, Liu T, Cypess AM, Gupta O, Holland WL, Gupta RK, Spray DC, Tanowitz HB, Cao L, Lynes MD, Tseng YH, Elmquist JK, Williams KW, Lin HV, Scherer PE. 2016. Connexin 43 Mediates White Adipose Tissue Beiging by Facilitating the Propagation of Sympathetic Neuronal Signals. Cell Metab 24: 420-33.

What is claimed is:

1. An engineered adeno-associated virus (AAV) vector comprising recombinant 2 (Rec2) capsid comprising one or more substitutions in the heparin binding loci; wherein the substitution confers tropism for neural tissue to the vector.

2. The engineered AAV vector of claim 1, wherein the one or more substitutions occurs at a residue corresponding to residues 588, 589, and/or 594 of SEQ ID NO: 1.

3. The engineered AAV vector of claim 2, wherein the one or more substitutions comprises Q588P, Q589L, Q589I, Q589V, Q589G, Q594L, Q594I, and/or Q594V.

4. The engineered AAV vector of claim 1, further comprising a first expression cassette comprising a regulatory element and a transgene operatively linked to a promoter and a second expression cassette comprising a tissue specific promoter operatively linked to a RNA silencing element that targets the regulatory element in the first expression cassette.

5. The method of claim 4, wherein the regulatory element of the first cassette is a woodchuck posttranscriptional regulatory element (WPRE) sequence.

6. A method of delivering a gene to neural tissue in the brain of a subject comprising administering to the subject the engineered AAV vector of claim 1 .

7. A method of treating a neurological disease in a subject comprising administering to the subject the engineered AAV vector of claim 1; wherein the engineered vector encodes a therapeutic agent.

8. The method of delivering a gene of claim 6, wherein the transgene or therapeutic agent comprises β-Galactosidase 1 (GLB1), Niemann-Pick C1 (NPC1), Apolipoprotein E (APOE), GD3 synthase, huntingtin (Htt), interleukin (IL)-10 (IL-10), Myelin Oligodendrocyte Glycoprotein (MOG), mitogen-activated protein kinase 8 interacting protein 3 (MAPKA8IP3), survival motor neuron (SMN) 1 (SMN1), SMN2, Cas9, β-Glucocerebrosidase (GBA), Sphingomyelin phosphodiesterase 1 (SMPD1), beta-hexosaminidase A (HEXA), nerve growth factor (NGF), brain-derived neurotrophic factor (BDNF), neurotrophin (NT) 3 (NT-3), NT-⅘, NT-6, Glial Cell Derived Neurotrophic Factor (GDNF), Ciliary Neurotrophic Factor (CNTF), Leukemia inhibitory factor (LIF), Insulin-like growth factor (IGF) 1 (IGF-1), β-fibroblast growth factor (FGF), neurturin, persephin, artemin, transforming growth factor (TGF) alpha (TGFα), TGFβ, IGF-2, platelet derived growth factor (PDGF), epidermal growth factor (EGF), cardiotropin, vascular endothelial growth factor (VEGF), Sonic hedgehog (SHH), bone morphogenic proteins (BMP), FGF20, Vasoactive Intestinal Peptide (VIP), pleiotrophin (PTN), Aromatic L-amino Acid Decarboxylase (AADC), TH, 5-hydroxytryptamine (5HT), hepatocyte growth factor (HGF), miRNA-222, miRNA-7, and/or miRNA-132.

9. The method of claim 7, wherein the neurological disease comprises Alzheimer’s disease, Parkinson’s disease and/or muscular dystrophy.

10. The method of delivering a gene claim 6 wherein the engineered AAV vector is administered retro-orbitally, intravenously, or via cerebrospinal fluid injection.

11. The method of treating a neurological disease of claim 7, wherein the transgene or therapeutic agent comprises β-Galactosidase 1 (GLB1), Niemann-Pick C1 (NPC1), Apolipoprotein E (APOE), GD3 synthase, huntingtin (Htt), interleukin (IL)-10 (IL-10), Myelin Oligodendrocyte Glycoprotein (MOG), mitogen-activated protein kinase 8 interacting protein 3 (MAPKA8IP3), survival motor neuron (SMN) 1 (SMN1), SMN2, Cas9, β-Glucocerebrosidase (GBA), Sphingomyelin phosphodiesterase 1 (SMPD1), beta-hexosaminidase A (HEXA), nerve growth factor (NGF), brain-derived neurotrophic factor (BDNF), neurotrophin (NT) 3 (NT-3), NT-⅘, NT-6, Glial Cell Derived Neurotrophic Factor (GDNF), Ciliary Neurotrophic Factor (CNTF), Leukemia inhibitory factor (LIF), Insulin-like growth factor (IGF) 1 (IGF-1), β-fibroblast growth factor (FGF), neurturin, persephin, artemin, transforming growth factor (TGF) alpha (TGFα), TGFβ, IGF-2, platelet derived growth factor (PDGF), epidermal growth factor (EGF), cardiotropin, vascular endothelial growth factor (VEGF), Sonic hedgehog (SHH), bone morphogenic proteins (BMP), FGF20, Vasoactive Intestinal Peptide (VIP), pleiotrophin (PTN), Aromatic L-amino Acid Decarboxylase (AADC), TH, 5-hydroxytryptamine (5HT), hepatocyte growth factor (HGF), miRNA-222, miRNA-7, and/or miRNA-132.

12. The method of treating a neurological disease of claim 7 wherein the engineered AAV vector is administered retro-orbitally, intravenously, or via cerebrospinal fluid injection.


SEQUENCE LISTING (6 sequences)

SEQ ID NO: 1 - protein, 738 aa, Artificial Sequence
  Synthetic construct
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEPSPQRSPDSSTGIGKTGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYQFEDVPFHSSYAHSQSLDRLMNPLIDQYLYYLSRTQSTGGTAGTQQLLFSQAGPNNMSAQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 2 - protein, 10 aa, Artificial Sequence
  Synthetic construct
CSWKYWFGEC

SEQ ID NO: 3 - protein, 10 aa, Artificial Sequence
  Synthetic construct
ATEEYGTVAD

SEQ ID NO: 4 - protein, 49 aa, Artificial Sequence
  Synthetic construct
CAGATAACTTGCAGCCGCTAAACACGGCTCCTCTAATTGGAACTGTCAA

SEQ ID NO: 5 - protein, 49 aa, Artificial Sequence
  Synthetic construct
TTGACAGTTCCAATTAGAGGAGCCGTGTTTAGCGGCTGCAAGTTATCTG

SEQ ID NO: 6 - protein, 738 aa, Artificial Sequence
  Synthetic construct
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEPSPQRSPDSSTGIGKTGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYQFEDVPFHSSYAHSQSLDRLMNPLIDQYLYYLSRTQSTGGTAGTQQLLFSQAGPNNMSAQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQPLNTAPLIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL